(19) United States
(12) Patent Application Publication
(10) Pub. No.: US 2023/0263721 A1
(43) Pub. Date: Aug. 24, 2023

(54) PROCESS FOR TREATING KERATIN FIBRES, COMPRISING THE APPLICATION OF A MAKEUP-REMOVING COMPOSITION, THE KERATIN FIBRES HAVING BEEN DYED BEFOREHAND

(71) Applicant: L'Oreal, Paris (FR)

(72) Inventors: Marine PROTAT-GERARDIN, SAINT OUEN (FR); Alexis LIARD, SAINT OUEN (FR); Jean-Daniel DEBAIN, SAINT OUEN (FR); Anne BANCHARAM, SAINT OUEN (FR)

(21) Appl. No.: 18/012,345

(22) PCT Filed: Jun. 23, 2021
(86) PCT No.: PCT/EP2021/067242

§ 371 Date: Dec. 22, 2022

(30) Foreign Application Priority Data
Jun. 23, 2020 (FR) FR2006554

(51) Int. Cl. A61K 8/898; A61Q 1/14; A61Q 5/02; A61K 8/891; A61K 8/06

(52) U.S. Cl. CPC A61K 8/898; A61Q 1/14; A61Q 5/02; A61K 8/891; A61K 8/068

(57) ABSTRACT

The present invention relates to a process for treating keratin fibres, in particular the hair, comprising the application of at least one makeup-removing composition to said keratin fibres, which have been dyed beforehand using at least one dye composition comprising at least one particular silicone of formula (I) and at least one colouring agent chosen from pigments, direct dyes and mixtures thereof, said makeup-removing composition comprising a) at least one alkaline agent.

The present invention relates to a process for treating keratin fibres, in particular the hair, comprising the application of at least one makeup-removing composition to said keratin fibres, which have been dyed beforehand using at least one dye composition comprising a particular silicone and at least one colouring agent chosen from pigments, direct dyes and mixtures thereof, said makeup-removing composition comprising at least one alkaline agent.

The invention also relates to a makeup-removing composition, which is in the form of a microemulsion, for removing a dye composition, comprising at least one hydrocarbon-based oil, at least one surfactant in a content of greater than or equal to 30% by weight relative to the total weight of the makeup-removing composition and at least one alkaline agent.

The invention also relates to the use of the makeup-removing composition according to the invention, or as used in the context of the process according to the invention, for removing a dye composition comprising at least one colouring agent chosen from pigments, direct dyes and mixtures thereof.

In the field of dyeing keratin fibres, in particular human keratin fibres, it is already known practice to dye keratin fibres via various techniques using direct dyes or pigments for non-permanent dyeing, or dye precursors for permanent dyeing.

There are essentially three types of process for dyeing the hair:

a) “permanent” dyeing, the function of which is to afford a substantial modification to the natural colour and which uses oxidation dyes which penetrate into the hair fibre and forms the dye via an oxidative condensation process;
b) non-permanent, semi-permanent or direct dyeing, which does not use the oxidative condensation process and withstands four or five shampoo washes; it consists in dyeing keratin fibres with dye compositions containing direct dyes;
c) temporary dyeing, which gives rise to a modification of the natural colour of the hair that remains from one shampoo wash to the next, and which serves to enhance or correct a shade that has already been obtained. It may also be likened to a “makeup” process.

For this last type of dyeing, it is known practice to use coloured polymers formed by grafting one or more dyes of azo, triphenylmethane, azine, indoamine or anthraquinone nature onto a polymer chain. These coloured polymers are not entirely satisfactory, notably as regards the homogeneity of the colouring obtained and its resistance, not to mention the problems associated with their manufacture and notably with their reproducibility.

Another dyeing method consists in using pigments. Specifically, the use of pigment on the surface of keratin fibres generally makes it possible to obtain visible colourings on dark hair, since the surface pigment masks the natural colour of the fibre. This dyeing method may also make it possible to have available dye compositions which have the advantage of producing a uniform coloured coating on keratin fibres, notably the hair, while at the same time forming a coat which withstands shampoo washing and the various attacking factors to which the hair may be subjected such as brushing and/or friction, without degradation of the hair.

However, no processes exist using makeup-removing compositions that are efficient for removing this type of temporary dye composition that is persistent with respect to shampoo washing.

A need thus remains for a process for efficiently removing makeup from keratin fibres which have been dyed beforehand using said dye compositions that are persistent with respect to shampoo washing.

Thus, the aim of the present invention is to develop a process for treating keratin fibres which makes it possible to improve the efficiency of makeup removal.

This aim is achieved with the present invention, one subject of which is a process for treating keratin fibres, in particular the hair, comprising the application of at least one makeup-removing composition to said keratin fibres, which have been dyed beforehand using at least one dye composition comprising:

at least one silicone of formula (I) below:

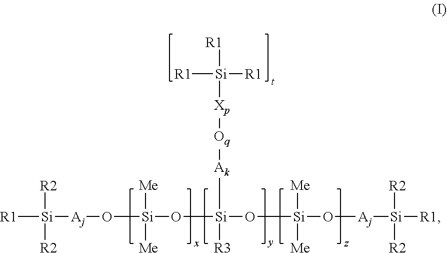

in which:
R1 independently represents a hydroxyl group or an alkoxy group containing from 1 to 2 carbon atoms or an alkyl group containing from 1 to 10 carbon atoms, preferably from 1 to 4 carbon atoms;
R2 independently represents an alkyl group containing from 1 to 10 carbon atoms, preferably from 1 to 4 carbon atoms, or an alkoxy group containing from 1 to 2 carbon atoms or a hydroxyl group or a monovalent radical of formula —C_{q}H_{2q}L in which q is a number ranging from 2 to 8, and L is an amino group, which is optionally quaternized, chosen from the following groups:
—N(R″)₂; —N+(R″)₃ A⁻; —NR″-Q-N(R″)₂ and —NR″-Q-N⁺(R″)₃A⁻,
in which R″, which may be identical or different, represents a hydrogen atom, a phenyl group, a benzyl group or a saturated monovalent hydrocarbon-based radical, for example a C₁-C₂₀ alkyl group; Q denotes a linear or branched group of formula CᵣH₂ᵣ, r being an integer ranging from 2 to 6, preferably from 2 to 4; and A⁻ represents a cosmetically acceptable anion, notably a halide such as fluoride, chloride, bromide or iodide;
R3 represents a hydroxyl group; an alkyl group containing from 1 to 10 carbon atoms, notably from 1 to 4 carbon atoms, said alkyl group being optionally substituted with at least one group chosen from a hydroxyl group (OH) or a thiol group (SH); a cycloalkyl group containing from 3 to 20 carbon atoms, notably from 5 to 6 carbon atoms, said cycloalkyl being optionally substituted with at least one group chosen from a hydroxyl group (OH) or a thiol group (SH); an alkoxy group containing from 1 to 2 carbon atoms, optionally substituted with at least one group chosen from a hydroxyl group (OH) or a thiol group (SH); an aryl group containing from 6 to 12 carbon atoms, optionally substituted with at least one group chosen from a hydroxyl group (OH) or a thiol group (SH); or a radical —(X)ₚ′—Si(R₂)₃ or a monovalent radical of formula —C_{q}C_{2q}L in which q is a number ranging from 2 to 8, and L is an amino group as described previously;
A represents a saturated divalent hydrocarbon-based radical containing 1 carbon atom;
X represents a hydrogen atom or a saturated divalent hydrocarbon-based radical containing 1 carbon atom;
p is an integer ranging from 0 to 6; p⁺ is an integer equal to 0 or 1 or 2 or 3;
k is an integer ranging from 0 to 6;
q denotes an integer equal to 0 or 1;
j denotes an integer equal to 0 or 1 or 2;
t denotes an integer equal to 0 or 1;
x denotes an integer ranging from 0 to 10, y denotes an integer ranging from 0 to 10, z denotes an integer ranging from 0 to 500 with x+z ranging from 0 to 500 and x+y+z ≥4;
it being understood that if X represents a hydrogen atom, then t=0 and p=1 and if p =0, then t=1 and q=1 and that at least one of the radicals R₁ or R₃ denotes a hydroxyl radical or an alkoxy radical containing from 1 to 2 carbon atoms or a radical —(X)_{p′}—Si(R₂)₃ or a monovalent radical of formula —C_{q}H_{2q}L in which q is a number ranging from 2 to 8, and L is an amino group as described previously; and
at least one colouring agent chosen from pigments, direct dyes and mixtures thereof,

said makeup-removing composition comprising at least one alkaline agent.

A subject of the invention is also a makeup-removing composition, which is in the form of a microemulsion, for removing a dye composition comprising at least one colouring agent chosen from pigments, direct dyes and mixtures thereof, comprising:

a) at least one alkaline agent;
b) at least one hydrocarbon-based oil; and
c) at least one surfactant in a content of greater than or equal to 30% by weight relative to the total weight of the makeup-removing composition.

The invention also relates to the use of the makeup-removing composition according to the invention, or as used in the context of the present invention, for removing a dye composition comprising at least one colouring agent chosen from pigments, direct dyes and mixtures thereof.

Other subjects, features, aspects and advantages of the invention will emerge even more clearly on reading the description and the examples that follow.

In the text hereinbelow, unless otherwise indicated, the limits of a range of values are included in that range, notably in the expressions “between” and “ranging from . . . to . . . ”.

The expression “at least one” used in the present description is equivalent to the expression “one or more”.

The terms “hydrocarbon-based radical” and “hydrocarbon-based chain” mean a radical or a chain comprising carbon and hydrogen atoms.

As indicated previously, the dye composition used in the context of the process according to the invention comprises at least one silicone of formula (I) as mentioned above.

More preferentially, the silicone(s) of formula (I) are such that:

R1 independently represents a hydroxyl group or an alkoxy group containing from 1 to 2 carbon atoms such as methoxy or ethoxy, or an alkyl group containing from 1 to 2 carbon atoms such as methyl;

R2 independently represents an alkyl group containing from 1 to 2 carbon atoms such as a methyl or an alkoxy group containing from 1 to 2 carbon atoms or a hydroxyl group;

R3 represents a hydroxyl group or an alkyl group containing from 1 to 10 carbon atoms and notably from 1 to 4 carbon atoms, or an alkoxy group containing from 1 to 2 carbon atoms or a radical —(X)_{p′}—Si(R₂)₃ or a monovalent radical of formula —C_{q}H_{2q}L in which q is a number ranging from 2 to 8, and L is an amino group chosen from the following groups:

—NN(R₄)₂;

N(R₄)—CH₂—CH₂—N(R₄)₂;

in which R₄ represents a hydrogen atom; a phenyl group; a benzyl group or a saturated monovalent hydrocarbon-based radical, for example a C₁-C₂₀ alkyl group;

A represents a saturated divalent hydrocarbon-based radical containing 1 carbon atom;

X represents a hydrogen atom or a hydrocarbon-based radical containing 1 carbon atom;

p denotes an integer ranging from 0 to 6, and p′ is an integer equal to 0 or 1 or 2 or 3;

j is equal to 0;

k is an integer equal to 0 or 1;

q denotes an integer equal to 0 or 1;

t denotes an integer equal to 0 or 1;

x denotes an integer ranging from 0 to 10, y denotes an integer ranging from 10 0 to 10, z denotes an integer ranging from 0 to 500 with x+z ranging from 0 to 500 and x+y+z≥4;

it being understood that if X represents a hydrogen atom, then t =0 and =1 and if p=0, then t=1 and q=1 and that at least one of the radicals R1 or R3 denotes a hydroxyl radical or an alkoxy radical containing from 1 to 2 carbon atoms or a monovalent radical of formula —C_{q}H_{2q}L as described previously.

More preferentially, the silicone(s) of formula (I) are such that:

R1 independently represents a hydroxyl group or an alkoxy group containing from 1 to 2 carbon atoms such as methoxy or ethoxy, or an alkyl group containing from 1 to 2 carbon atoms such as methyl;

R2 independently represents an alkyl group containing from 1 to 2 carbon atoms such as a methyl or an alkoxy group containing from 1 to 2 carbon atoms or a hydroxyl group;

R3 represents a monovalent radical of formula —C_{q}H_{2q}L in which q is a number ranging from 2 to 8 and L is an amino group chosen from the following groups:

—N(R₄)₂;

—N(R₄)—CH₂—CH₂—N(R₄)₂;

in which R₄ represents a hydrogen atom; a phenyl group; a benzyl group or a saturated monovalent hydrocarbon-based radical, for example a C₁-C₂₀ alkyl group;

A represents a saturated divalent hydrocarbon-based radical containing 1 carbon atom;

X represents a hydrogen atom, and p denotes an integer equal to 1;

j is equal to 0;

k is equal to 1;

q denotes an integer equal to 0;

t denotes an integer equal to 0;

x denotes an integer ranging from 0 to 10, y denotes an integer ranging from 0 to 10, z denotes an integer ranging from 0 to 500 with x+z ranging from 0 to 500 and x+y+z≥4;

it being understood that at least one of the radicals R1 or R3 denotes a hydroxyl radical or an alkoxy radical containing from 1 to 2 carbon atoms or a monovalent radical of formula —C_{q}H_{2q}L as described previously.

The silicone(s) of formula (I) used in the context of the invention may be chosen from:

the compounds of formula (Ia) below:

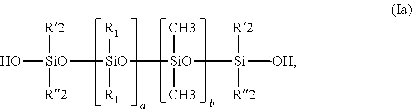

in which:

R₁, which may be identical or different, independently represents a hydroxyl group; an alkyl group containing from 1 to 4 carbon atoms, preferably from 1 to 2 carbon atoms such as a methyl, or an alkoxy group containing from 1 to 2 carbon atoms;
R′₂ and R″₂ independently represent an alkyl group containing from 1 to 4 carbon atoms, notably from 1 to 2 carbon atoms such as a methyl, or a hydroxyl group;
a denotes an integer ranging from 0 to 10, b denotes an integer ranging from 0 to 500 with a+b≥4;
the compounds of formula (Ib) below:

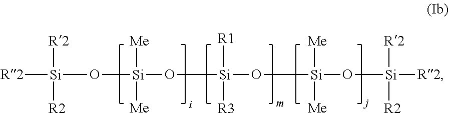

in which:
   R₁ independently represents an alkyl group containing from 1 to 4 carbon atoms, and most particularly a methyl, or an alkoxy group containing from 1 to 2 carbon atoms;
   R₂, R′₂ and R″₂, which may be identical or different, independently represent an alkyl group containing from 1 to 4 carbon atoms and in particular from 1 to 2 carbon atoms, or a hydroxyl group or an alkoxy group containing from 1 to 2 carbon atoms;
   R₃ represents a group —Xp′—Si(OR₄)₃ with R₄ representing an alkyl group containing from 1 to 2 carbon atoms, X representing a saturated divalent hydrocarbon-based radical containing 1 carbon atom, and p′ denoting an integer ranging from 1 to 3;
   m denotes an integer ranging from 0 to 3;
   i denotes an integer ranging from 0 to 10, j denotes an integer ranging from 0 to 500 and i+j ranging from 0 to 510 with i+j≥4;
the compounds of formula (Ic) below:

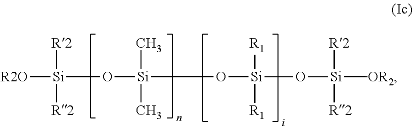

in which:
   R₁, which may be identical or different, represents an alkyl group containing from 1 to 4 carbon atoms, preferably from 1 to 2 carbon atoms, such as a methyl; or an alkoxy group containing from 1 to 2 carbon atoms;
   R₂ independently represents an alkyl group containing from 1 to 2 carbon atoms such as a methyl or ethyl;
   R′₂ and R″₂, which may be identical or different, independently represent an alkyl group containing from 1 to 4 carbon atoms, more preferentially from 1 to 2 carbon atoms, or an alkoxy group containing from 1 to 2 carbon atoms such as a methoxy or ethoxy;
   i denotes an integer ranging from 0 to 10, n denotes an integer ranging from 0 to 500 with n+i ranging from 0 to 510 with n+i≥4;

the compounds of formula (Id) below:

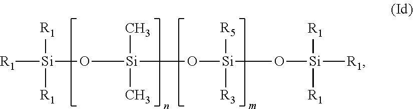

in which:

m and n are numbers such that the sum (m+n) ranges from 1 to 500, n denoting a number ranging from 0 to 499, and m denoting a number ranging from 1 to 500;
R₁, which may be identical or different, independently represents an alkyl group containing from 1 to 4 carbon atoms, more preferentially from 1 to 2 carbon atoms, for example a methyl; or a hydroxyl group or an alkoxy group containing from 1 to 2 carbon atoms;
R₃ represents a monovalent radical of formula —C_{q}H_{2q}L in which q is a number ranging from 2 to 8 and L is an amino group chosen from the following groups:
—N(R₄)₂;
—N(R₄)—CH₂—CH₂—N(R₄)₂;
in which R₄ represents a hydrogen atom; a phenyl group; a benzyl group or a saturated monovalent hydrocarbon-based radical, for example a C₁-C₂₀ alkyl group; and
R₅ represents a hydroxyl group or an alkyl group containing from 1 to 2 carbon atoms, for example a methyl, or an alkoxy group containing from 1 to 2 carbon atoms;
and mixtures thereof.

Among the silicones of formula (Ia), mention may be made of polydimethylsiloxanes (PDMS) bearing hydroxyl end functions, such as the compounds sold by the company Shin-Etsu under the name KF-9701 or X-21-5841, or those sold by the company Sigma-Aldrich under the reference 481939 (Mn˜550, ˜25 cSt), 481955 (˜65 cSt), or 481963 (˜750 cSt).

Among the silicones of formula (Ib), mention may be made of polydimethylsiloxanes (PDMS) bearing trialkoxysilane side functions, such as those sold by the company Siltech under the name Silmer TMS C50.

Among the silicones of formula (Ic), mention may be made of polydimethylsiloxanes (PDMS) bearing trialkoxysilane end functions, such as those sold by the company Power Chemical under the name SiSiB® PF2110, or those sold by the company Siltech under the name Silmer TMS Di-10 or Silmer TMS Di-50.

Among the silicones of formula (Id) mention may be made of the compound having the trade name KF 857, sold by the company Shin-Etsu, or the compound having the trade name KF 862, sold by the company Shin-Etsu.

Preferably, the silicone(s) are chosen from the silicones of formulae (Ia) and (Id) and mixtures thereof.

More preferentially, the silicone(s) of formula (I) used in the context of the invention are chosen from the compounds of formula (Ia) in which:

R₁ independently represents an alkyl group containing from 1 to 4 carbon atoms, preferably from 1 to 2 carbon atoms such as a methyl;
R′₂ and R″₂ independently represent an alkyl group containing from 1 to 4 carbon atoms, more particularly from 1 to 2 carbon atoms such as methyl;
b denotes an integer ranging from 0 to 10, a denotes an integer ranging from 0 to 5 with a+b≥4.

The silicone(s) of formula (I) may be present in a total amount ranging from 0.1% to 30% by weight, preferably from 1% to 25% by weight and more preferentially from 1% to 20% by weight relative to the total weight of the dye composition.

Alkoxysilane

The dye composition may also comprise at least one alkoxysilane chosen from the compounds of formula (II) below, oligomers thereof and/or mixtures thereof:

R¹ₓSi(OR²)₄₋ₓ (II),

in which:

R¹ represents an alkoxy group containing from 1 to 10 carbon atoms, a linear or branched, saturated or unsaturated, cyclic or acyclic C₁ to C₂₂ and notably C₁ to C₂₀ hydrocarbon-based radical, which may be substituted with at least one group chosen from a hydroxyl group (OH); a thiol group; an amino group NH_{2′}; an alkylamino group NHR in which R denotes a linear or branched alkyl radical containing from 1 to 20 carbon atoms, notably from 1 to 10 carbon atoms; an alkoxy group containing from 1 to 10 carbon atoms; a cycloalkyl containing from 3 to 40 carbon atoms; an aryl containing from 6 to 30 carbon atoms; R¹ possibly being interrupted with at least one heteroatom chosen from O, S, NH or a carbonyl group (CO);
R² represents a hydrogen atom or an alkyl group containing from 1 to 20 carbon atoms, preferably from 2 to 6 carbon atoms;
x denotes an integer ranging from 1 to 3;

R¹ and R² may be identical or different.

The term “oligomer” means compound(s) including at least two silicon atoms, obtained by oligomerization or polymerization of the compounds of formula (II).

Preferably, R¹ represents an alkoxy group containing from 1 to 10 carbon atoms, such as an ethoxy, or R¹ represents a linear or branched, saturated C₁ to C₂₂ and notably C₁ to C₂₀ hydrocarbon-based radical, which is preferably linear, said hydrocarbon-based radical possibly being substituted with at least one amino group NH₂ or alkylamino group NHR, in which R denotes a linear or branched alkyl containing from 1 to 20 carbon atoms, notably from 1 to 10 carbon atoms.

More preferentially, R1 represents a saturated linear C1 to C6 hydrocarbon-based radical, which may be substituted with an amino group NH₂.

Preferably, R² represents an alkyl group containing from 1 to 10 carbon atoms, preferably from 2 to 6 carbon atoms, more preferentially an ethyl.

The alkoxysilane(s) of formula (II), the oligomers thereof and/or mixtures thereof may be chosen from:

the compounds of formula (IIa) and/or (IIb) below, oligomers thereof, alone or as a mixture:

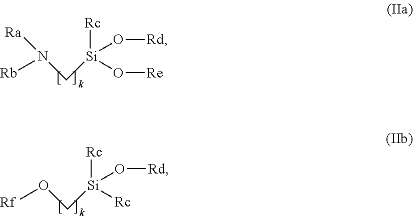

in which:

Ra and Rb, which may be identical or different, represent a hydrogen atom; an alkyl group containing from 1 to 20 carbon atoms, preferably from 1 to 10 carbon atoms and notably from 1 to 4 carbon atoms; a cycloalkyl group containing from 3 to 20 carbon atoms; an aryl group containing from 6 to 12 carbon atoms; an aminoalkyl group containing from 1 to 20 carbon atoms;
Rc represents a hydrogen atom; an alkyl group containing from 1 to 20 carbon atoms, preferably from 1 to 10 carbon atoms, more preferentially from 1 to 4 carbon atoms and in particular from 1 to 2 carbon atoms such as a methyl; an alkoxy group containing from 1 to 10 carbon atoms, preferably from 1 to 4 carbon atoms and in particular from 1 to 2 carbon atoms such as an ethoxy, or an alkylaryl group containing from 7 to 12 carbon atoms;
Rd and Re, which may be identical or different, represent an alkyl group containing from 1 to 10 carbon atoms, preferably from 1 to 4 carbon atoms, in particular from 1 to 2 carbon atoms, such as an ethyl;
k denotes an integer ranging from 0 to 5, preferably ranging from 0 to 3;
Rf represents a hydrogen atom; an alkyl group containing from 1 to 20 carbon atoms, preferably from 1 to 10 carbon atoms and notably from 1 to 4 carbon atoms; or a group of formula (III) below:

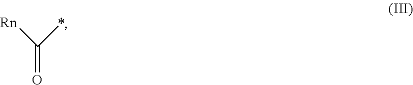

in which Rn represents a hydroxyl group (OH); an alkyl group containing from 1 to 20 carbon atoms, preferably from 1 to 10 carbon atoms, preferably a methyl.

Among the alkoxysilanes of formula (IIa), the oligomers thereof and/or mixtures thereof, mention may notably be made of 3-aminopropyltriethoxysilane (APTES), 3-aminopropylmethyldiethoxysilane (APMDES) and N-cyclohexyl-aminomethyltriethoxysilane.

APTES may be purchased, for example, from the company Dow Corning under the name Xiameter OFS-6011 Silane or from the company Momentive Performance Materials under the name Silsoft A-1100 or from the company Shin-Etsu under the name KBE-903.

The compounds of formula (IIa) may also denote Dynasylan SIVO 210 or Dynasylan 1505 sold by the company Evonik.

N-Cycloheylaminomethyltriethoxysilane may be purchased, for example, from the company Wacker under the name Geniosil XL 926.

Among the alkoxysilanes of formula (IIb), the oligomers thereof and/or mixtures thereof, mention may notably be made of tetraethoxysilane (TEOS), methyltriethoxysilane (MTES), dimethyldiethoxysilane (DMDES), diethyldiethoxysilane, dipropyldiethoxysilane, propyltriethoxysilane, isobutyltriethoxysilane, phenyltriethoxysilane, phenylmethyldiethoxysilane, diphenyldiethoxysilane, benzyltriethoxysilane, benzylmethyldiethoxysilane, dibenzyldiethoxysilane, acetoxymethyltriethoxysilane and mixtures thereof.

TEOS may be purchased, for example, from the company Evonik under the name Dynasylan® A or Dynasylan® A SQ. MTES may be purchased, for example, from the company Evonik under the name Dynasylan® MTES. DMDES may be purchased, for example, from the company Gelest under the reference SID3404.0.

Preferably, the alkoxysilane(s) chosen from the compounds of formula (II), the oligomers thereof and/or mixtures thereof are chosen from 3-aminopropyltriethoxysilane (APTES), 3-aminopropylmethyldiethoxysilane (APMDES), N-cyclohexylaminomethyltriethoxysilane, tetraethoxysilane (TEOS), methyltriethoxysilane (MTES), dimethyldiethoxysilane (DMDES), diethyldiethoxysilane, dipropyldiethoxysilane, propyltriethoxysilane, isobutyltriethoxysilane, phenyltriethoxysilane, phenylmethyldiethoxysilane, diphenyldiethoxysilane, benzyltriethoxysilane, benzylmethyldiethoxysilane, dibenzyldiethoxysilane, acetoxymethyltriethoxysilane and mixtures thereof, more preferentially 3-aminopropyltriethoxysilane (APTES), tetraethoxysilane (TEOS) and mixtures thereof.

Preferably, the alkoxysilane(s) of formula (II), the oligomers thereof and/or mixtures thereof are chosen from the compounds of formula (IIa) below:

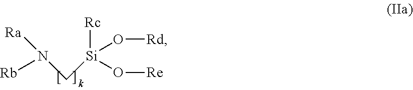

in which:

Ra and Rb, which are identical, represent a hydrogen atom or Ra denotes a hydrogen atom and Rb denotes a C5-C6 cycloalkyl radical such as cyclohexyl;

Rc represents an alkyl group containing from 1 to 10 carbon atoms, notably from 1 to 4 carbon atoms and in particular from 1 to 2 carbon atoms, preferably a methyl, or an alkoxy group containing from 1 to 4 carbon atoms, preferably from 1 to 2 carbon atoms, preferably an ethoxy;
Rd and Re, which may be identical or different, represent an alkyl group containing from 1 to 10 carbon atoms, preferably from 1 to 4 carbon atoms, such as an ethyl;
k denotes an integer ranging from 1 to 3 and more particularly 1 or 3.

Preferentially, the compounds of formula (IIa) are such that Ra and Rb represent a hydrogen atom, Rc represents an ethoxy group, Rd and Re are identical and represent an ethyl and k is equal to 3.

Preferably, the alkoxysilane of formula (II), the oligomers thereof and/or mixtures thereof, is 3-aminopropyltriethoxysilane (APTES).

According to another preferred embodiment, the alkoxysilane(s) of formula (II), the oligomers thereof and/or mixtures thereof are chosen from the compounds of formula (IIb) below:

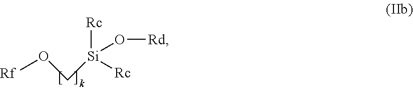

in which

Rc represents an alkoxy group containing from 1 to 10 carbon atoms, preferably from 1 to 4 carbon atoms and in particular from 1 to 2 carbon atoms, such as an ethoxy;
Rd represents an alkyl group containing from 1 to 10 carbon atoms, preferably from 1 to 4 carbon atoms, in particular from 1 to 2 carbon atoms, such as an ethyl;
k denotes an integer ranging from 0 to 3, preferably equal to 0;
Rf represents a hydrogen atom or an alkyl group containing from 1 to 10 carbon atoms and notably from 1 to 4 carbon atoms such as an ethyl.

According to another more preferred embodiment, the alkoxysilane of formula (II), the oligomers thereof and/or mixtures thereof, is tetraethoxysilane (TEOS).

The alkoxysilane(s) of formula (II), oligomers thereof and/or mixtures thereof may be present in a total amount ranging from 0.1% to 30% by weight, preferably from 0.5% to 25% by weight and better still from 0.5% to 20% by weight, relative to the 25 total weight of the dye composition.

Colouring Agent

The process according to the invention comprises a dye composition comprising at least one colouring agent chosen from pigments, direct dyes and mixtures thereof.

Preferably, the dye composition according to the invention comprises one or more pigments.

The term “pigment” refers to any pigment that gives colour to keratin materials. Their solubility in water at 25° C. and at atmospheric pressure (760 mmHg) is less than 0.05% by weight, and preferably less than 0.01%.

The pigments that may be used are notably chosen from the organic and/or mineral pigments known in the art, notably those described in Kirk-Othmer's Encyclopedia of Chemical Technology and in Ullmann's Encyclopedia of Industrial Chemistry.

They may be natural, of natural origin, or non-natural.

These pigments may be in pigment powder or paste form. They may be coated or uncoated.

The pigments may be chosen, for example, from mineral pigments, organic pigments, lakes, pigments with special effects such as nacres or glitter flakes, and mixtures thereof.

The pigment may be a mineral pigment. The term “mineral pigment” refers to any pigment that satisfies the definition in Ullmann's encyclopaedia in the chapter on inorganic pigments. Among the mineral pigments that are useful in the present invention, mention may be made of iron oxides, chromium oxides, manganese violet, ultramarine blue, chromium hydrate, ferric blue and titanium oxide.

The pigment may be an organic pigment. The term “organic pigment” refers to any pigment that satisfies the definition in Ullmann' s encyclopaedia in the chapter on organic pigments.

The organic pigment may notably be chosen from nitroso, nitro, azo, xanthene, pyrene, quinoleine, quinoline, anthraquinone, triphenylmethane, fluorane, phthalocyanine, metal-complex, isoindolinone, isoindoline, quinacridone, perinone, perylene, diketopyrrolopyrrole, indigo, thioindigo, dioxazine, triphenylmethane and quinophthalone compounds.

In particular, the white or coloured organic pigments may be chosen from carmine, carbon black, aniline black, azo yellow, quinacridone, phthalocyanine blue, the blue pigments codified in the Colour Index under the references CI 42090, 69800, 69825, 74100, 74160, the yellow pigments codified in the Colour Index under the references CI 11680, 11710, 19140, 20040, 21100, 21108, 47000, 47005, the green pigments codified in the Colour Index under the references CI 61565, 61570, 74260, the orange pigments codified in the Colour Index under the references CI 11725, 45370, 71105, the red pigments codified in the Colour Index under the references CI 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names:

Cosmenyl Yellow IOG: Yellow 3 pigment (CI 11710);
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1 184 426. These composite pigments may be composed notably of particles including a mineral core, at least one binder for attaching the organic pigments to the core, and at least one organic pigment which at least partially covers the core.

The organic pigment may also be a lake. The term “lake” means dyes adsorbed onto insoluble particles, the assembly thus obtained remaining insoluble during use.

The mineral substrates onto which the dyes are adsorbed are, for example, alumina, silica, calcium sodium borosilicate or calcium aluminium borosilicate and aluminium.

Among the dyes, mention may be made of carminic acid. Mention may also be made of the dyes known under the following names: D&C Red 21 (CI 45 380),

D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semi-transparent or transparent shade.

Several types of pigments with special effects exist: those with a low refractive index, such as fluorescent or photochromic pigments, and those with a higher refractive index, such as nacres, interference pigments or glitter flakes.

Examples of pigments with special effects that may be mentioned include nacreous pigments such as mica covered with titanium or with bismuth oxychloride, coloured nacreous pigments such as mica covered with titanium and with iron oxides, mica covered with iron oxide, mica covered with titanium and notably with ferric blue or with chromium oxide, mica covered with titanium and with an organic pigment as defined previously, and also nacreous pigments based on bismuth oxychloride. Nacreous pigments that may be mentioned include the nacres Cellini sold by BASF (mica-TiO₂-lake), Prestige sold by Eckart (mica-TiO₂), Prestige Bronze sold by Eckart (mica-Fe2O3) and Colorona sold by Merck (mica-TiO₂—Fe₂O₃).

Mention may also be made of the gold-coloured nacres sold notably by the company BASF under the name Brilliant gold 212G (Timica), Gold 222C (Cloisonne), Sparkle gold (Timica), Gold 4504 (Chromalite) and Monarch gold 233X (Cloisonne); the bronze nacres sold notably by the company Merck under the name Bronze fine (17384) (Colorona) and Bronze (17353) (Colorona) and by the company BASF under the name Super bronze (Cloisonne); the orange nacres sold notably by the company BASF under the name Orange 363C (Cloisonne) and Orange MCR 101 (Cosmica) and by the company Merck under the name Passion orange (Colorona) and Matte orange (17449) (Microna); the brown nacres sold notably by the company BASF under the name Nu-antique copper 340XB (Cloisonne) and Brown CL4509 (Chromalite); the nacres with a copper tint sold notably by the company BASF under the name Copper 340A (Timica); the nacres with a red tint sold notably by the company Merck under the name Sienna fine (17386) (Colorona); the nacres with a yellow tint sold notably by the company BASF under the name Yellow (4502) (Chromalite); the red nacres with a gold tint sold notably by the company BASF under the name Sunstone G012 (Gemtone); the pink nacres sold notably by the company BASF under the name Tan opale G005 (Gemtone); the black nacres with a gold tint sold notably by the company BASF under the name Nu antique bronze 240 AB (Timica), the blue nacres sold notably by the company Merck under the name Matte blue (17433) (Microna), the white nacres with a silvery tint sold notably by the company Merck under the name Xirona Silver, and the golden-green pink-orange nacres sold notably by the company Merck under the name Indian summer (Xirona), and mixtures thereof.

Still as examples of nacres, mention may also be made of particles including a borosilicate substrate coated with titanium oxide.

Particles comprising a glass substrate coated with titanium oxide are notably sold under the name Metashine MC108ORY by the company Toyal.

Finally, examples of nacres that may also be mentioned include polyethylene terephthalate glitter flakes, notably those sold by the company Meadowbrook

Inventions under the name Silver 1P 0.004X0.004 (silver glitter flakes). It is also possible to envisage multilayer pigments based on synthetic substrates, such as alumina, silica, calcium sodium borosilicate, calcium aluminium borosilicate and aluminium.

The pigments with special effects may also be chosen from reflective particles, i.e. notably from particles whose size, structure, notably the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, highlight points that are visible to the naked eye, i.e. more luminous points that contrast with their environment, making them appear to sparkle.

The reflective particles may be selected so as not to significantly alter the colouring effect generated by the colouring agents with which they are combined, and more particularly so as to optimize this effect in terms of colour rendition. They may more particularly have a yellow, pink, red, bronze, orange, brown, gold and/or coppery colour or tint.

These particles may have varied forms and may notably be in platelet or globular form, in particular in spherical form.

The reflective particles, whatever their form, may or may not have a multilayer structure and, in the case of a multilayer structure, may have, for example, at least one layer of uniform thickness, notably of a reflective material.

When the reflective particles do not have a multilayer structure, they may be composed, for example, of metal oxides, notably titanium or iron oxides obtained synthetically.

When the reflective particles have a multilayer structure, they may include, for example, a natural or synthetic substrate, notably a synthetic substrate at least partially coated with at least one layer of a reflective material, notably of at least one metal or metallic material. The substrate may be made of one or more organic and/or mineral materials.

More particularly, it may be chosen from glasses, ceramics, graphite, metal oxides, aluminas, silicas, silicates, notably aluminosilicates and borosilicates, and synthetic mica, and mixtures thereof, this list not being limiting.

The reflective material may include a layer of metal or of a metallic material.

Reflective particles are notably described in JP-A-09188830, JP-A-10158450, JP-A-10158541, JP-A-07258460 and JP-A-05017710.

Again as an example of reflective particles including a mineral substrate coated with a layer of metal, mention may also be made of particles including a silver-coated borosilicate substrate.

Particles with a silver-coated glass substrate, in the form of platelets, are sold under the name Microglass Metashine REFSX 2025 PS by the company Toyal. Particles with a glass substrate coated with nickel/chromium/molybdenum alloy are sold under the names Crystal Star GF 550 and GF 2525 by this same company.

Use may also be made of particles comprising a metal substrate, such as silver, aluminium, iron, chromium, nickel, molybdenum, gold, copper, zinc, tin, magnesium, steel, bronze or titanium, said substrate being coated with at least one layer of at least one metal oxide, such as titanium oxide, aluminium oxide, iron oxide, cerium oxide, chromium oxide, silicon oxides and mixtures thereof.

Examples that may be mentioned include aluminium powder, bronze powder or copper powder coated with SiO₂ sold under the name Visionaire by the company Eckart.

Mention may also be made of pigments with an interference effect which are not attached to a substrate, such as liquid crystals (Helicones HC from Wacker) or interference holographic glitter flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

The variety of pigments that may be used in the present invention makes it possible to obtain a wide range of colours, and also particular optical effects such as metallic effects or interference effects.

The size of the pigment used in the composition according to the present invention is generally between 10 nm and 200 μm, preferably between 20 nm and 80 μm and more preferentially between 30 nm and 50 μm.

The pigments may be dispersed in the composition by means of a dispersant.

The dispersant serves to protect the dispersed particles against their agglomeration or flocculation. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of C8 to C20 fatty acid and of polyols such as glycerol or diglycerol are used, such as poly(12-hydroxystearic acid) stearate with a molecular weight of approximately 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the composition may be surface-treated with an organic agent.

Thus, the pigments that have been surface-treated beforehand, which are useful in the context of the invention, are pigments that have totally or partially undergone a surface treatment of chemical, electronic, electrochemical, mechanochemical or mechanical nature, with an organic agent such as those described notably in Cosmetics and Toiletries, February 1990, Vol. 105, pages 53-64, before being dispersed in the composition in accordance with the invention. These organic agents may be chosen, for example, from waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminium salts of fatty acids, for example aluminium stearate or laurate; metal alkoxides; polyethylene; (meth)acrylic polymers, for example polymethyl methacrylates; polymers and copolymers containing acrylate units; alkanolamines; silicone compounds, for example silicones, notably polydimethylsiloxanes; organofluorine compounds, for example perfluoroalkyl ethers; luorosilicone compounds.

The surface-treated pigments that are useful in the composition may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is notably described in patent U.S. Pat. No. 4,578,266.

An organic agent covalently bonded to the pigments will preferably be used.

The agent for the surface treatment may represent from 0.1% to 50% by weight relative to the total weight of the surface-treated pigment, preferably from 0.5% to 30% by weight and even more preferentially from 1% to 20% by weight relative to the total weight of the surface-treated pigment.

Preferably, the surface treatments of the pigments are chosen from the following treatments:

a PEG-silicone treatment, for instance the AQ surface treatment sold by LCW;
a methicone treatment, for instance the SI surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a magnesium myristate treatment, for instance the MM surface treatment sold by LCW;
an aluminium dimyristate treatment, such as the MI surface treatment sold by Miyoshi;
a perfluoropolymethyl isopropyl ether treatment, for instance the FHC surface treatment sold by LCW;
an isostearyl sebacate treatment, for instance the HS surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrogenosiloxane/perfluoroalkyl phosphate treatment, for instance the FS01 surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
an acrylate copolymer treatment, for instance the APD surface treatment sold by Daito;
a perfluoroalkyl phosphate/isopropyl titanium triisostearate treatment, for instance the PF+ITT surface treatment sold by Daito.

According to a particular embodiment of the invention, the dispersant is present with organic or mineral pigments in submicron-sized particulate form in the dye composition.

The term “submicron” or “submicronic” refers to pigments having a particle size that has been micronized by a micronization method and having a mean particle size of less than a micrometre (μm), in particular between 0.1 and 0.9 μm, and preferably between 0.2 and 0.6 μm.

According to one embodiment, the dispersant and the pigment(s) are present in an amount (dispersant:pigment), according to a weight ratio, of between 1:4 and 4:1, particularly between 1.5:3.5 and 3.5:1 or better still between 1.75:3 and 3:1.

The dispersant(s) may thus have a silicone backbone, such as silicone polyether and dispersants of amino silicone type other than the alkoxysilanes described previously. Among the suitable dispersants that may be mentioned are:

amino silicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones bearing carboxyl groups such as X-22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to a particular embodiment, the dispersant(s) are of amino silicone type other than the alkoxysilanes described previously and are cationic.

Preferably, the pigment(s) are chosen from mineral, mixed mineral-organic or organic pigments.

In one variant of the invention, the pigment(s) are organic pigments, preferentially organic pigments surface-treated with an organic agent chosen from silicone compounds. In another variant of the invention, the pigment(s) are mineral pigments.

Direct Dye

The dye composition used in the context of the process according to the invention may comprise one or more direct dyes.

The term “direct dye” means natural and/or synthetic dyes, other than oxidation dyes. These are dyes that will spread superficially on the fibre.

They may be ionic or nonionic, preferably cationic or nonionic. Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (III) and (IV) and the azo cationic dyes (V) and (VI) below:

Het⁺—N(Ra)—N═C(Rb)—Ar, Q- (III),

Het⁺-C(Ra)═N—N(Rb)—Ar, (IV),

Het⁺-N═N—Ar, Q- (V),

Ar⁺—N═N—Ar″, Q- (VI),

in which formulae (III) to (VI):

Het+ represents a cationic heteroaryl radical, preferentially bearing an endocyclic cationic charge, such as imidazolium, indolium or pyridinium, which is optionally substituted, preferentially with at least one (C₁-C₈) alkyl group such as methyl; Ar+ represents an aryl radical, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially with one or more electron-donating groups such as i) optionally substituted (C₁-C₈)alkyl, ii) optionally substituted (C₈₁-C₈₈)alkoxy, iii) (di)(C₈-C₈)(alkyl)amino optionally substituted on the alkyl group(s) with a hydroxyl group, iv) aryl(C₈₁-C₈₈)alkylamino, v) optionally substituted N-(C₁-C₈)alkyl-N-aryl(C₈-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₈₁-C₈)(alkyl)amino, (C₁-C₈)alkoxy or phenyl groups;
Ra and Rb, which may be identical or different, represent a hydrogen atom or a (C₁-C₈)alkyl group, which is optionally substituted, preferentially with a hydroxyl group;
or else the substituent Ra with a substituent of Het+ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q- represents an organic or mineral anionic counterion, such as a halide or an alkyl sulfate.

In particular, mention may be made of the azo and hydrazono direct dyes bearing an endocyclic cationic charge of formulae (III) to (VI) as defined previously, more particularly, the cationic direct dyes bearing an endocyclic cationic charge described in patent applications WO 95/15144, WO 95/01772 and EP 714 954, preferentially the following direct dyes:

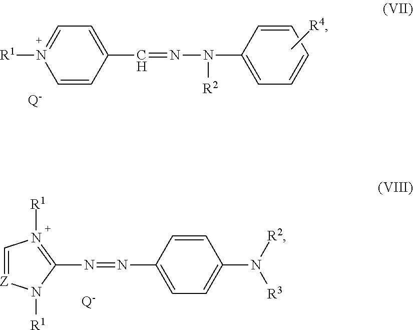

in which formulae (VII) and (VIII):

R¹ represents a (C₁-C₄)alkyl group such as methyl;
R² and R³, which may be identical or different, represent a hydrogen atom or a (C₁-C₄)alkyl group, such as methyl; and
R₄ represents a hydrogen atom or an electron-donating group such as optionally substituted (C₁-C₈)alkyl, optionally substituted (C₁-C₈)alkoxy, or (di)(Cᵢ-C₈)(alkyl)amino optionally substituted on the alkyl group(s) with a hydroxyl group; in particular, R⁴ is a hydrogen atom;
Z represents a CH group or a nitrogen atom, preferentially CH,
Q- is an anionic counterion as defined previously, in particular a halide, such as chloride, or an alkyl sulfate, such as methyl sulfate or mesyl.

In particular, the dyes of formulae (V) and (VI) are chosen from Basic Red 51, Basic Yellow 87 and Basic Orange 31 or derivatives thereof with Q′ being an anionic counterion as defined previously, particularly halide such as chloride, or an alkyl sulfate such as methyl sulfate or mesyl.

The direct dyes may be chosen from anionic direct dyes. The anionic direct dyes of the invention are dyes commonly referred to as “acid” direct dyes owing to their affinity for alkaline substances. The term “anionic direct dye” means any direct dye including in its structure at least one CO₂R or SO₃R substituent with R denoting a hydrogen atom or a cation originating from a metal or an amine, or an ammonium ion. The anionic dyes may be chosen from direct nitro acid dyes, azo acid dyes, azine acid dyes, triarylmethane acid dyes, indoamine acid dyes, anthraquinone acid dyes, indigoid dyes and natural acid dyes.

As acid dyes that are useful for the invention, mention may be made of the dyes of formulae (IX), (IX′), (X), (X′), (XI), (XI′), (XII), (XII′), (XIII), (XIV), (XV) and (XVI) below:

a) the diaryl anionic azo dyes of formula (IX) or (IX′):

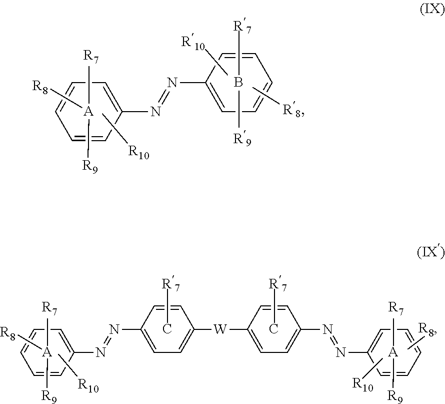

in which formulae (IX) and (IX′):
R₇, R₈, R₉, R₁₀, R′₇, R′₈, R′₉ and R′₁₀, which may be identical or different, represent a hydrogen atom or a group chosen from:
alkyl;
alkoxy, alkylthio;
hydroxyl, mercapto;
nitro, nitroso;
R°—C(X)—X′—, R°—X′—C(X)—, R°X′—C(X)—X″— with R° representing a hydrogen atom or an alkyl or aryl group; X, X′ and X″, which may be identical or different, representing an oxygen or sulfur atom, or NR with R representing a hydrogen atom or an alkyl group;
(O)₂S(O—)—, M⁺ with M⁺ representing a hydrogen atom or a cationic counterion;
(O)CO⁻—, M⁺ with M⁺ as defined previously;
R″—S(O)₂—, with R″ representing a hydrogen atom or an alkyl, aryl, (di)(alkyl)amino or aryl(alkyl)amino group; preferentially a phenylamino or phenyl group;
R′″—S(O)₂—X′— with R′″ representing an optionally substituted alkyl or aryl group, X′ as defined previously;
(di)(alkyl)amino;
aryl(alkyl)amino optionally substituted with one or more groups chosen from i) nitro; ii) nitroso; iii) (O)₂S(O⁻)—, M⁺ and iv) alkoxy with M⁺ as defined previously;
optionally substituted heteroaryl; preferentially a benzothiazolyl group;
cycloalkyl, notably cyclohexyl;
Ar—N═N— with Ar representing an optionally substituted aryl group; preferentially a phenyl optionally substituted with one or more alkyl, (O)₂S(O⁻)—, M⁺ or phenylamino groups;
or alternatively two contiguous groups R₇ with R₈ or R₈ with R₉ or R₉ with R₁₀ together form a fused benzo group A′; and R′₇ with R′₈ or R′₈ with R′9 or R′₉ with R′₁₀ together form a fused benzo group B′; with A′ and B′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R°—C(X)—X′—; viii) R°—X′—C(X)—; ix) R°—X′—C(X)—X″—; x) Ar—N═N— and xi) optionally substituted aryl(alkyl)amino; with M⁺, R°, X, X′, X″ and Ar as defined previously;
W represents a sigma bond σ, an oxygen or sulfur atom, or a divalent radical i) —NR— with R as defined previously, or ii) methylene—C(Ra)(Rb)— with Ra and Rb, which may be identical or different, representing a hydrogen atom or an aryl group, or alternatively

Ra and Rb form, together with the carbon atom that bears them, a Spiro cycloalkyl; preferentially, W represents a sulfur atom or Ra and Rb together form a cyclohexyl; it being understood that formulae (IX) and (IX′) comprise at least one sulfonate radical (O)₂S(O⁻)—, N⁺ or one carboxylate radical (O)CO⁻—, M⁺ on one of the rings A, A′, B, B′ or C; preferentially sodium sulfonate.

As examples of dyes of formula (IX), mention may be made of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment Red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3, Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2, Food Yellow 3 or 15 Sunset Yellow;

and, as examples of dyes of formula (IX′), mention may be made of: Acid Red 111, Acid Red 134, Acid Yellow 38;

b) the pyrazolone anionic azo dyes of formulae (X) and (X′):

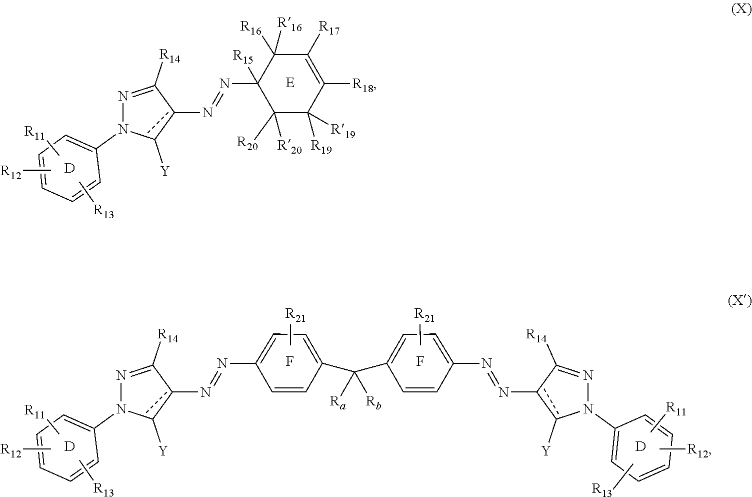

in which formulae (X) and (X′):

R₁₁, R₁₂ and RR₁₃, which may be identical or different, represent a hydrogen or halogen atom, an alkyl group or —(O)₂S(O⁻), N⁺ with M⁺ as defined previously;
R₁₄ represents a hydrogen atom, an alkyl group or a group —C(O)O⁻, M⁺ with M⁺ as defined previously;
R₁₅ represents a hydrogen atom;
R₁₆ represents an oxo group, in which case R′₁₆ is absent, or alternatively R₁₅ with R₁₆ together form a double bond;
R₁₇ and R₁₈, which may be identical or different, represent a hydrogen atom, or a group chosen from:
(O)₂S(O⁻)—, M⁺ with M⁺ as defined previously;
Ar—O—S(O)₂— with Ar representing an optionally substituted aryl group; preferentially a phenyl optionally substituted with one or more alkyl groups;
R₁₉ and R₂₀ together form either a double bond, or a benzo group D′, which is optionally substituted;
R′₁₆, R′₁₉ and R′₂₀, which may be identical or different, represent a hydrogen atom or an alkyl or hydroxyl group;
R₂₁ represents a hydrogen atom or an alkyl or alkoxy group;
Ra and Rb, which may be identical or different, are as defined previously;

preferentially, Ra represents a hydrogen atom and Rb represents an aryl group;

Y represents either a hydroxyl group or an oxo group; [symbol]
represents a single bond when Y is an oxo group; and represents a double bond when Y represents a hydroxyl group;
it being understood that formulae (X) and (X′) comprise at least one sulfonate radical (O)₂S(O⁻)—, M⁺ or one carboxylate radical —C(O)O⁻, M⁺ on one of the rings D or E; preferentially sodium sulfonate.

As examples of dyes of formula (X), mention may be made of: Acid Red 195, Acid Yellow 23, Acid Yellow 27, Acid Yellow 76, and as examples of dyes of formula (X′), mention may be made of: Acid Yellow 17;

c) the anthraquinone dyes of formulae (XI) and (XI′):

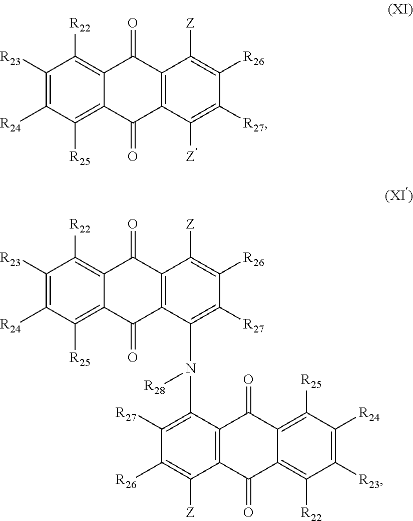

in which formulae (XI) and (XI′):

R₂₂, R₂₃, R₂₄, R₂₅, R₂₆ and R₂₇, which may be identical or different, represent a hydrogen or halogen atom, or a group chosen from:
alkyl;
hydroxyl, mercapto;
alkoxy, alkylthio;
optionally substituted aryloxy or arylthio, preferentially substituted with one or more groups chosen from alkyl and (O)₂S(O⁻), M⁺ with M⁺ as defined previously;
aryl(alkyl)amino optionally substituted with one or more groups chosen from alkyl and (O)₂S(O—, M⁺ with M⁺ as defined previously;
(di)(alkyl)amino;
(di)(hydroxyalkyl)amino;
(O)₂S(O⁻)—, M⁺ with M⁺ as defined previously;
Z′ represents a hydrogen atom or a group NR₂₈R₂₉ with R₂₈ and R₂₉, which may be identical or different, representing a hydrogen atom or a group chosen from:
alkyl;
polyhydroxyalkyl such as hydroxyethyl;
aryl optionally substituted with one or more groups, particularly i) alkyl such as methyl, n-dodecyl, n-butyl; ii) (O)₂S(O⁻)—, M⁺ with M⁺ as defined previously; iii) R°—C(X)—X′—, R°—X′—C(X)-, R°—X′—C(X)—X″— with R°, X, X′ and X″ as defined previously, preferentially R° represents an alkyl group;
cycloalkyl; notably cyclohexyl;
Z represents a group chosen from hydroxyl and NR′₂₈R′₂₉ with R′₂₈ and R′₂₉, which may be identical or different, representing the same atoms or groups as R₂₈ and R₂₉ as defined previously;
it being understood that formulae (XI) and (XI′) comprise at least one sulfonate radical (O)₂S(O⁻)—, M⁺ or one carboxylate radical C(O)O⁻, M⁺; preferentially sodium sulfonate.

As examples of dyes of formula (XI), mention may be made of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT Violet No. 2; and, as an example of a dye of formula (XI′), mention may be made of: Acid Black 48;

d) the nitro dyes of formulae (XII) and (XII′):

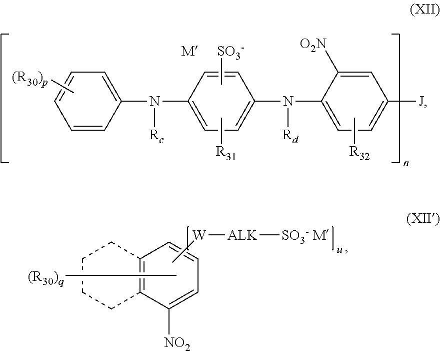

in which formulae (XII) and (XII′):

R₃₀, R₃₀ and R₃₂, which may be identical or different, represent a hydrogen or halogen atom, or a group chosen from:
alkyl;
alkoxy optionally substituted with one or more hydroxyl groups, alkylthio optionally substituted with one or more hydroxyl groups;
hydroxyl, mercapto;
nitro, nitroso;
polyhaloalkyl;
R°—C(X)—X′—, R°—X′—C(X)—, R°—X′—C(X)—X″— with R°, X, X′ and X″ as defined previously;
(O)₂S(O⁻)—, M⁺ with M⁺ as defined previously;
—(O)CO—, M⁺ with M⁺ as defined previously;
(di)(alkyl)amino;
(di)(hydroxyalkyl)amino;
heterocycloalkyl such as piperidino, piperazino or morpholino; in particular, R₃₀, R₃₁ and R₃₂ represent a hydrogen atom;
Rc and Rd, which may be identical or different, represent a hydrogen atom or an alkyl group;
W is as defined previously; W particularly represents an —NH— group;
ALK represents a linear or branched divalent C₁-C₆ alkylene group; in particular, ALK represents a —CH₂—CH₂— group;
n is 1 or 2;
p represents an integer inclusively between 1 and 5;
q represents an integer inclusively between 1 and 4;
u is 0 or 1;
when n is 1, J represents a nitro or nitroso group; particularly nitro;
when n is 2, J represents an oxygen or sulfur atom, or a divalent radical —S(O)ₘ— with m representing an integer 1 or 2; preferentially, J represents an —SO₂— radical;
M′ represents a hydrogen atom or a cationic counterion;

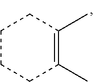

which may be present or absent, represents a benzo group optionally substituted with one or more groups R₃₀ as defined previously;
it being understood that formulae (XII) and (XII′) comprise at least one sulfonate radical (O)₂S(O⁻)—, M⁺ or one carboxylate radical C(O)O⁻, M⁺; preferentially sodium sulfonate.

As examples of dyes of formula (XII), mention may be made of: Acid Brown 13 and Acid Orange 3; as examples of dyes of formula (XII′), mention may be made of: Acid Yellow 1, the sodium salt of 2,4-dinitro-1-naphthol-7-sulfonic acid, 2-piperidino-5-nitrobenzenesulfonic acid, 2-(4′-N,N-(2″-hydroxyethyl)amino-2′-nitro)anilineethanesulfonic acid, 4-β-hydroxyethylamino-3-nitrobenzenesulfonic acid; EXT D&C Yellow 7;

e) the triarylmethane dyes of formula (XIII):

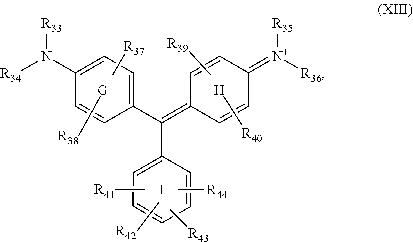

in which formula (XIII):

R₃₃, R₃₄, R₃₅ and R₃₆, which may be identical or different, represent a hydrogen atom or a group chosen from alkyl, optionally substituted aryl and optionally substituted arylalkyl; particularly an alkyl and benzyl group optionally substituted with a group (O)ₘS(O⁻)—, M⁺ with M⁺ and m as defined previously;
R₃₇, R₃₈, R₃₉, R₄₀, R₄₁, R₄₂, R₄₃ and R₄₄, which may be identical or different, represent a hydrogen atom or a group chosen from:
alkyl;
alkoxy, alkylthio;
(di)(alkyl)amino;
hydroxyl, mercapto;
nitro, nitroso;
R°—C(X)—X′—, R°—X′—C(X)—, R°—X′-C(X)—X″— with R° representing a hydrogen atom or an alkyl or aryl group; X, X′ and X″, which may be identical or different, representing an oxygen or sulfur atom, or NR with R representing a hydrogen atom or an alkyl group;
—(O)₂S(O⁻), M⁺ with M⁺ representing a hydrogen atom or a cationic counterion;
(O)CO⁻—, M⁺ with M⁺ as defined previously;
or alternatively two contiguous groups R₄₁ with R₄₂ or R₄₂ with R₄₃ or R₄₃ with R₄₄ together form a fused benzo group: I′; with I′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R°—C(X)—X′—; viii) R°—X′—C(X)— and ix) R°—X′—C(X)—X″—; with M⁺, R°, X, X′ and X″ as defined previously;
in particular, R₃₇ to R₄₀ represent a hydrogen atom, and R₄₁ to R₄₄, which may be identical or different, represent a hydroxyl group or (O)₂S(O⁻)—, M⁺; and when R₄₃ with R₄₄ together form a benzo group, it is preferentially substituted with an (O)₂S(O⁻)— group;
it being understood that at least one of the rings G, H, I or I′ comprises at least one sulfonate radical (O)₂S(O)— or one carboxylate radical —C(O)O⁻; preferentially 25 sulfonate.

As examples of dyes of formula (XIII), mention may be made of: Acid Blue 1; Acid Blue 3; Acid Blue 7, Acid Blue 9; Acid Violet 49; Acid Green 3; Acid Green 5 and Acid Green 50.

f) the xanthene-based dyes of formula (XIV):

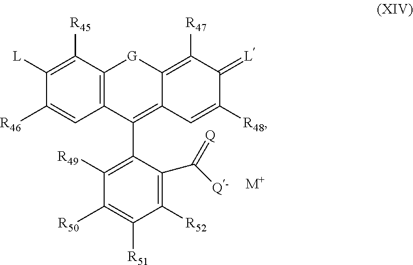

in which formula (XIV):

R₄₅, R₄₆, R₄₇ and R₄₈, which may be identical or different, represent a hydrogen or halogen atom;
R₄₉, R₅₀, R₅₁ and R₅₂, which may be identical or different, represent a hydrogen or halogen atom, or a group chosen from:
alkyl;
alkoxy, alkylthio;
hydroxyl, mercapto;
nitro, nitroso;
(O)₂S(O⁻)—, M⁺ with M⁺ representing a hydrogen atom or a cationic counterion;
(O)CO⁻—, M⁺ with M⁺ as defined previously; particularly, R₄₉, R₅₀, R₅₁ and R₅₂ represent a hydrogen or halogen atom;
G represents an oxygen or sulfur atom or a group NRe with Re as defined previously; particularly, G represents an oxygen atom;
L represents an alkoxide O⁻, M⁺; a thioalkoxide S⁻,M⁺ or a group NRf, with Rf representing a hydrogen atom or an alkyl group, and M⁺ as defined previously; M⁺ is particularly sodium or potassium;
L′ represents an oxygen or sulfur atom or an ammonium group: N+RfRg, with Rf and Rg, which may be identical or different, representing a hydrogen atom or an optionally substituted alkyl or aryl group; L′ particularly represents an oxygen atom or a phenylamino group optionally substituted with one or more alkyl or (O)ₘS(O⁻)—, M⁺ groups with m and M⁺ as defined previously;
Q and Q′, which may be identical or different, represent an oxygen or sulfur atom; particularly, Q and Q′ represent an oxygen atom;
M⁺ is as defined previously.

As examples of dyes of formula (XIV), mention may be made of: Acid Yellow 73; Acid Red 51; Acid Red 52; Acid Red 87; Acid Red 92; Acid Red 95; Acid Violet 9;

g) the indole-based dyes of formula (XV):

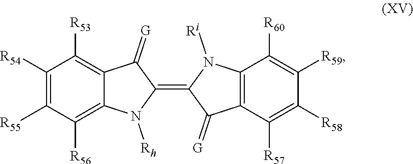

in which formula (XV):

R₅₃, R₅₄, R₅₅, R₅₆,R₅₇, R₅₈, R₅₉ and R₆₀, which may be identical or different, represent a hydrogen atom or a group chosen from:
alkyl;
alkoxy, alkylthio;
hydroxyl, mercapto;
nitro, nitroso;
R°—C(X)—X′—, R°—X′—C(X)—, R°—X′—C(X)—X″— with R° representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined previously;
G represents an oxygen or sulfur atom or a group NRe with Re as defined previously; particularly, G represents an oxygen atom;
Ri and Rh, which may be identical or different, represent a hydrogen atom or an alkyl group;
it being understood that formula (XIII) comprises at least one sulfonate radical (O)₂S(O⁻)—, M⁺ or one carboxylate radical —C(O)O⁻, M⁺; preferentially sodium sulfonate.

As examples of dyes of formula (XV), mention may be made of: Acid Blue 74;

g) the quinoline-based dyes of formula (XVI):

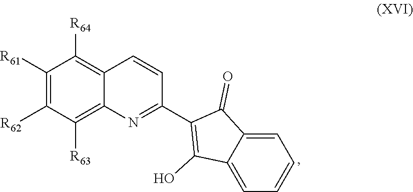

in which formula (XVI):

R₆₁ represents a hydrogen or halogen atom or an alkyl group;
R₆₂, R₆₃ and R₆₄, which may be identical or different, represent a hydrogen atom or a group (O)₂S(O⁻)—, M⁺ with M⁺ representing a hydrogen atom or a cationic counterion;

or alternatively R₆₁ with R₆₂, or R₆₁ with R₆₄, together form a benzo group optionally substituted with one or more groups (O₂S(O⁻)—, M⁺ with M⁺ representing a hydrogen atom or a cationic counterion;

it being understood that formula (XVI) comprises at least one sulfonate radical (O)₂S(O⁻)—, M⁺, preferentially sodium sulfonate.

As examples of dyes of formula (XVI), mention may be made of: Acid Yellow 2, Acid Yellow 3 and Acid Yellow 5.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and notably henna-based poultices or extracts.

Preferably, the direct dyes are chosen from anionic direct dyes.

The colouring agent(s) may be present in a total content ranging from 0.001% to 20% by weight and preferably from 0.005% to 15% by weight relative to the total weight of the dye composition.

The pigment(s) may be present in a total content ranging from 0.05% to 20% by weight, preferably from 0.1% to 15% by weight and better still from 0.5% to 10% by weight, relative to the total weight of the dye composition.

The direct dye(s) may be present in a total content ranging from 0.001% to 10% by weight relative to the total weight of the composition, preferably from 0.005% to 5% by weight relative to the total weight of the dye composition.

Organic Solvents

The dye composition used in the context of the process according to the invention may comprise one or more organic solvents.

Examples of organic solvents that may be mentioned include lower C₁-C₄ alkanols, such as ethanol and isopropanol; polyols and polyol ethers, for instance 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

Preferably, the composition comprises one or more organic solvents chosen from C₁-C₄ lower alkanols, more preferentially ethanol.

The organic solvents may be present in a total amount inclusively between 0.1% and 60% by weight approximately relative to the total weight of the dye 20 composition, preferably between 1% and 50% by weight and more preferentially inclusively between 5% and 45% by weight relative to the total weight of the dye composition.

Additives

The dye composition used in the context of the process according to the invention may contain any adjuvant or additive usually used.

Among the additives that may be contained in the dye composition, mention may be made of reducing agents, thickeners, softeners, antifoams, moisturizers, UV-screening agents, peptizers, solubilizers, fragrances, anionic, cationic, nonionic or amphoteric surfactants, proteins, vitamins, polymers, preserving agents, oils, waxes and mixtures thereof.

Preferably, the dye composition comprises a pH agent such as hydrochloric acid.

The dye composition may notably be in the form of a suspension, a dispersion, a gel, an emulsion, notably an oil-in-water (O/W) or water-in-oil (W/O) emulsion, or a multiple emulsion (W/O/W or polyol/O/W or O/W/O), in the form of a cream, a mousse, a stick, a dispersion of vesicles, notably of ionic or nonionic lipids, or a two-phase or multi-phase lotion.

A person skilled in the art may select the appropriate presentation form, and also the method for preparing it, on the basis of his general knowledge, taking into account firstly the nature of the constituents used, notably their solubility in the support, and secondly the intended application of the composition.

According to a preferred embodiment, the dye composition used in the context of the process according to the invention may comprise a silicone of formula (Ia), 3-aminopropyltriethoxysilane (APTES) and pigments.

As indicated previously, the process for treating keratin fibres, in particular the hair, comprises the application of at least one makeup-removing composition to said keratin fibres, which have been dyed beforehand using at least one dye composition as defined above, said makeup-removing composition comprising at least one alkaline agent.

Alkaline Agent

Preferably, the alkaline agent(s) may be chosen from organic alkaline agents and inorganic alkaline agents.

Preferably, the organic alkaline agent(s) are chosen from organic amines, the pKb of which at 25° C. is less than 12, more preferentially less than 10 and even more advantageously less than 6. It should be noted that it is the pKb corresponding to the function which has the highest basicity. In addition, the organic amines do not comprise any alkyl or alkenyl fatty chains comprising more than ten carbon atoms.

The organic alkaline agent(s) are preferably chosen from alkanolamines, in particular mono-, di- or tri-hydroxy(C₁-C₆)alkylamines, such as 2-amino-2-methylpropanol, monoethanolamine, oxyethylenated and/or oxypropylenated ethylenediamines, amino acids, the polyamines of formula (I) below, and mixtures thereof:

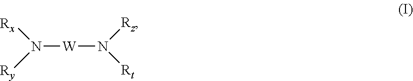

in which formula (I) W is a divalent C₁ to C₆ alkylene radical optionally substituted with one or more hydroxyl groups or a C₁ to C₆ alkyl radical, and/or optionally interrupted with one or more heteroatoms such as O, or NRᵤ; Rₓ, R_{y}, R_{z}, Rₜ, and Rᵤ, which may be identical or different, represent a hydrogen atom, a C₁ to C₆ alkyl or C₁ to C₆ hydroxyalkyl or C₁ to C₆ aminoalkyl radical.

Examples of amines of formula (I) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁ to C₈ alkyl groups bearing one or more hydroxyl radicals.

Organic amines chosen from alkanolamines such as monoalkanolamines, dialkanolamines or trialkanolamines comprising one to three identical or different C₁ to C₄ hydroxyalkyl radicals are in particular suitable for performing the invention.

Among the compounds of this type, mention may be made of monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethylamino)methane.

More particularly, the amino acids that may be used are of natural or synthetic origin, in their L, D or racemic form, and include at least one acid function chosen more particularly from carboxylic acid, sulfonic acid, phosphonic acid and phosphoric acid functions. The amino acids may be in neutral or ionic form.

As amino acids that may be used in the present invention, mention may notably be made of aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine and valine.

Advantageously, the amino acids are basic amino acids comprising an additional amine function optionally included in a ring or in a ureido function.

Such basic amino acids are preferably chosen from those corresponding to formula (II) below, and also salts thereof:

R—CH₂—CH(NH₂)—C(O-—H (II),

in which formula (II) R represents a group chosen from imidazolyl, preferably imidazolyl-4-yl; aminopropyl; aminoethyl; —(CH₂)₂N(H)—C(O)—NH₂; and —(CH₂)₂—N(H)—C(NH)—NH₂.

The organic amine may also be chosen from organic amines of heterocyclic type. Besides histidine that has already been mentioned in the amino acids, mention may in particular be made of pyridine, piperidine, imidazole, triazole, tetrazole and benzimidazole.

The organic amine may also be chosen from amino acid dipeptides. As amino acid dipeptides that may be used in the present invention, mention may notably be made of carnosine, anserine and balenine.

The organic amine may also be chosen from compounds including a guanidine function. As amines of this type that may be used in the present invention, besides arginine, which has already been mentioned as an amino acid, mention may be made notably of creatine, creatinine, 1,1-dimethylguanidine, 1,1-diethylguanidine, glycocyamine, metformin, agmatine, n-amidinoalanine, 3-guanidinopropionic acid, 4-guanidinobutyric acid, 2-([amino(imino)methyl]amino)ethane-1-sulfonic acid and guanidine carbonate.

Among the inorganic alkaline agents that may be used in the makeup-removing composition used in the context of the process according to the invention, mention may be made of ammonium hydroxide (also known as aqueous ammonia), mineral hydroxides, alkali metal or alkaline-earth metal silicates, metasilicates, phosphates, hydrogen phosphates, carbonates and hydrogen carbonates, and in particular alkali metal silicates or metasilicates, notably sodium silicate and sodium metasilicate.

The mineral hydroxides may be chosen from alkali metal, alkaline-earth metal and transition metal hydroxides. Examples of mineral hydroxides that may be mentioned include sodium hydroxide, lithium hydroxide, calcium hydroxide, magnesium hydroxide, barium hydroxide, strontium hydroxide, manganese hydroxide and zinc hydroxide.

Among the mineral hydroxides, sodium hydroxide is preferred.

Preferably, the alkaline agent(s) that are useful in the invention are chosen from alkanolamines, alkali metal silicates and metasilicates, mineral hydroxides and mixtures thereof.

Even more preferentially, the alkaline agent(s) that are useful in the invention are chosen from monoethanolamine, sodium hydroxide, sodium silicate, sodium metasilicate and mixtures thereof, better still from monoethanolamine and sodium metasilicate.

Advantageously, the content of the alkaline agent(s) ranges from 0.01% to 15% by weight, preferably from 0.1% to 10% by weight, more preferentially from 0.5% to 5% by weight and even more preferentially from 0.5% to 2% by weight, relative to the total weight of the makeup-removing composition.

Microemulsion

Preferably, the makeup-removing composition used in the context of the process according to the invention is in the form of a microemulsion.

The term “microemulsion” means a thermodynamically stable, microscopically heterogeneous and macroscopically homogeneous mixture of two 15 mutually immiscible liquid substances, such as an oily phase and an aqueous phase.

Microemulsions may be of oil-in-water type (O/W), i.e. droplets of oil dissolved in the form of direct micelles swollen in an aqueous continuous phase, or of water-in-oil type (W/O), i.e. droplets of water dissolved in the form of reverse micelles swollen in an oily continuous phase, or alternatively of bicontinuous type, i.e. in the form of structures in which the water and the oil are codis solved, the water and the oil being able to be considered simultaneously as being the continuous phase or the dispersed phase.

Microemulsions are to be distinguished from nanoemulsions, which are thermodynamically unstable dispersions of oil or water droplets in a continuous aqueous or oily phase.

Microemulsions are formed by simple mixing of the various constituents, without the need for a large energy input.

The microemulsion according to a preferred embodiment of the invention has the advantage of being able to be prepared without heating, without a large energy input, which facilitates its industrial manufacture and, moreover, of being able to use heat-sensitive active agents which it may be difficult to incorporate into standard emulsions or nanoemulsions when the manufacturing process requires a heating step liable to degrade them.

In addition, microemulsions generally have a particular microstructure formed from microdroplets whose size is such that light passes through them without being scattered, and as such the appearance of this composition is transparent or translucent, whereas the appearance of a standard emulsion is opaque.

Furthermore, the microemulsion according to a preferred embodiment of the invention has the advantage of being stable, even when the amount of fatty substance present is high, in particular when it is greater than or equal to 15% by weight relative to the weight of the composition.

Furthermore, relative to a standard emulsion or a nanoemulsion, the microemulsion according to a preferred embodiment of the invention allows better penetration of the active agents into the keratin fibre due to the small size of its microdroplets and thus of constituting a better vector for the active agents. Furthermore, a microemulsion according to a preferred embodiment of the invention may contain a larger amount of active agents, and in particular of active agents that are difficult to dissolve.

In particular, this makes it possible to reduce the amount of active agents and/or of dye composition required and/or the application time of the composition on the fibres to dye or bleach them.

In the case where the microemulsion according to a preferred embodiment of the invention is in the form of droplets dispersed in a continuous phase, the numerical mean size of the droplets of the dispersed phase of the microemulsions according to a preferred embodiment of the invention is preferably less than 100 nm, even more preferentially less than 50 nm and better still between 1 and 50 nm.

The numerical mean size of the particles may be determined in particular according to the known method of quasi-elastic light scattering. As a machine that may be used for this determination, mention may be made of the Brookhaven brand machine equipped with an SX 200 optical bed (with a 532 nm laser) and a BI 9000 correlator. This machine gives a measurement of the mean diameter by photon correlation spectroscopy (PCS), which makes it possible to determine the numerical mean diameter from the polydispersity factor, which is also measured by the machine.

The microemulsion may be prepared via standard processes for preparing microemulsions, which are well known to those skilled in the art, in particular following the production of phase diagrams for determining the formation domain of the microemulsion.

After passing through a centrifuge, the microemulsion according to a preferred embodiment of the invention returns spontaneously to its initial thermodynamic equilibrium at a given temperature. Moreover, the microemulsion remains stable even after two months of storage at 45° C.

Preferably, the makeup-removing composition is in the form of a water-in-oil microemulsion.

Preferably, the makeup-removing composition comprises an oily phase.

The oily phase comprises at least one hydrocarbon-based oil and may also comprise one or more fatty substances other than the hydrocarbon-based oils as described below.

Hydrocarbon-Based Oil

Preferably, the makeup-removing composition used in the context of the process according to the invention comprises b) at least one hydrocarbon-based oil. Preferably, the oily phase of the makeup-removing composition comprises at least one hydrocarbon-based oil.

The term “oil” means a fatty substance that is liquid at room temperature (25° C.) and at atmospheric pressure (760 mmHg or 1.013×105 Pa).

The term “fatty substance” means an organic compound that is insoluble in water at ordinary temperature (25° C.) and at atmospheric pressure (760 mmHg or 1.013×10⁵ Pa) (solubility of less than 5%, preferably less than 1% and even more preferentially less than 0.1%). They bear in their structure at least one hydrocarbon-based chain including at least 6 carbon atoms or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

The term “hydrocarbon-based oil” means an oil formed essentially from, or even constituted of, carbon and hydrogen atoms, and possibly oxygen and nitrogen atoms, and not containing any silicon or fluorine atoms. It may contain alcohol, ester, ether, carboxylic acid, amine and/or amide groups.

Mention may be made of volatile hydrocarbon-based oils and non-volatile hydrocarbon-based oils.

Preferably, the makeup-removing composition comprises at least one non-volatile hydrocarbon-based oil.

The term “non-volatile oil” means an oil that remains on the skin or the keratin fibre at room temperature and atmospheric pressure. More precisely, a non-volatile oil has an evaporation rate strictly less than 0.01 mg/cm2/min.

To measure this evaporation rate, 15 g of oil or oil mixture to be tested are placed in a crystallizing dish 7 cm in diameter, placed on a balance that is in a large chamber of about 0.3 m³ which is temperature-regulated, at a temperature of 25° C., and hygrometry-regulated, at a relative humidity of 50%. The liquid is allowed to evaporate freely, without stirring it, while providing ventilation by means of a fan (Papst-Motoren, reference 8550 N, rotating at 2700 rpm) placed in a vertical position above the crystallizing dish containing said oil or said mixture, the blades being directed towards the crystallizing dish, 20 cm away from the bottom of the crystallizing dish. The mass of oil remaining in the crystallizing dish is measured at regular intervals. The evaporation rates are expressed in mg of oil evaporated per unit area (cm2) and per unit time (minutes).

The non-volatile hydrocarbon-based oil is preferably chosen from:

hydrocarbon-based oils of plant origin, such as triglycerides formed from fatty acid esters of glycerol, the fatty acids of which may have chain lengths ranging from C₄ to C₂₈, these fatty acids possibly being linear or branched, and saturated or unsaturated; these oils are notably wheatgerm oil, sunflower oil, beauty-leaf oil, grapeseed oil, sesame seed oil, corn oil, apricot oil, castor oil, shea oil, avocado oil, olive oil, soybean oil, sweet almond oil, rapeseed oil, cottonseed oil, hazelnut oil, macadamia oil, jojoba oil, palm oil, alfalfa oil, poppy oil, pumpkin oil, marrow oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by the company Stéarinerie Dubois or those sold under the names Miglyol 810®, 812® and 818® by the company Sasol;
synthetic ethers containing from 10 to 40 carbon atoms, such as dicaprylyl ether;
linear or branched hydrocarbons, of mineral or synthetic origin, such as petroleum jelly, polybutenes, polydecenes and squalane;
synthetic esters and/or ethers such as the oils of formula R₁COOR₂ and/or R₁OR₂ in which R₁ represents a linear or branched fatty acid residue including from 1 to 40 carbon atoms and R₂ represents a, notably branched, hydrocarbon-based chain containing from 1 to 40 carbon atoms, on condition that the total carbon number of R₂+R₂≥10, for instance purcellin oil (cetostearyl octanoate), isopropyl myristate, isopropyl palmitate, butyl stearate, Cie to Cis alkyl benzoate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, 2-diethylhexyl succinate, isostearyl isostearate, alkyl or polyalkyl octanoates, decanoates or ricinoleates such as propylene glycol dioctanoate; hydroxylated esters such as isostearate lactate and diisostearyl malate; lanolic acid, oleic acid, lauric acid or stearic acid esters; glyceryl or diglyceryl triisostearate; and pentaerythritol esters;
fatty alcohols that are liquid at room temperature, bearing a branched and/or unsaturated carbon-based chain containing from 12 to 26 carbon atoms, for instance cetanol, octyldodecanol, stearyl alcohol, linoleyl alcohol, linolenyl alcohol, isostearyl alcohol, oleyl alcohol, 2-hexyldecanol, 2-butyloctanol or 2-undecylpentadecanol;
fatty acids such as myristic acid, palmitic acid, stearic acid, behenic acid, oleic acid, linoleic acid, linolenic acid and isostearic acid; and
mixtures thereof.

Preferably, the makeup-removing composition comprises a total content of non-volatile hydrocarbon-based oil(s) ranging from 10% to 95% by weight, preferably from 20% to 80% by weight and more preferentially from 20% to 60% by weight, relative to the weight of the makeup-removing composition.

The term “volatile oil” means an oil that can evaporate on contact with the skin in less than one hour, at room temperature and atmospheric pressure. The volatile oil is a cosmetic volatile oil, which is liquid at room temperature. More specifically, a volatile oil has an evaporation rate of between 0.01 and 200 mg/cm^{2/}min, limits inclusive (see the above protocol for measuring the evaporation rate).

The volatile hydrocarbon-based oils that are suitable for use in the invention may be chosen from hydrocarbon-based oils containing from 7 to 16 carbon atoms. The volatile hydrocarbon-based oils may be chosen from branched alkanes and linear alkanes.

Volatile hydrocarbon-based oils containing from 7 to 16 carbon atoms that may notably be mentioned include branched C₈-C₁₆ alkanes, for instance C₈-C₁₆ isoalkanes (also known as isoparaffins), isododecane, isodecane, isohexadecane and, for example, the oils sold under the trade name Isopar or Permethyl, branched C₈-C₁₆ esters, for instance isohexyl neopentanoate, and mixtures thereof. Preferably, the volatile hydrocarbon-based oil containing from 7 to 16 carbon atoms is chosen from isododecane, isodecane and isohexadecane and mixtures thereof, and is notably isododecane.

As volatile hydrocarbon-based oils that may be used for the purposes of the present invention, mention may be made of linear alkanes, preferably of plant origin, comprising from 7 to 15 carbon atoms, in particular from 9 to 14 carbon atoms and more particularly from 11 to 13 carbon atoms.

As examples of linear alkanes that are suitable for use in the invention, mention may be made of n-heptane (C7), n-octane (C8), n-nonane (C9), n-decane (C10), n-undecane (C11), n-dodecane (C12), n-tridecane (C13), n-tetradecane (C14) and n-pentadecane (C15), and mixtures thereof, and in particular the mixture of n-undecane (C11) and n-tridecane (C13) described in Example 1 of patent application WO 2008/155 059 by the company Cognis. Mention may also be made of n-dodecane (C12) and n-tetradecane (C14) sold by Sasol under the references, respectively, Parafol 12-97 and Parafol 14-97, and also mixtures thereof.

In this case, the makeup-removing composition may comprise a total content of volatile hydrocarbon-based oil(s), when they are present, ranging from 0.1% to 10% by weight and better still from 0.5% to 5% by weight, relative to the total weight of the makeup-removing composition.

The hydrocarbon-based oil(s) may also be defined according to their molecular weight and their solubility parameter δa.

Preferably, a hydrocarbon-based oil has a molecular weight of less than or equal to 400 g/mol and a solubility parameter δa ranging from 2 to 15 J^{0.5}/cm^{1.5}.

The definition and calculation of the solubility parameters in the Hansen three-dimensional solubility space are described in the article by C. M. Hansen: “The three-dimensional solubility parameters”, J. Paint Technol. 39, 105 (1967).

According to this Hansen space:

δD characterizes the London dispersion forces derived from the formation of dipoles induced during molecular impacts;

δp characterizes the Debye interaction forces between permanent dipoles and also the Keesom interaction forces between induced dipoles and permanent dipoles;

δh characterizes the specific interaction forces (such as hydrogen bonding, acid/base, donor/acceptor, etc.);

δa is determined by the equation: δa=(δp²+δh²)^{1/2}.

The parameters δd, δp, δh and δa are expressed in (J/cm³)^{1/2}.

The global solubility parameter δ according to the Hansen solubility space is defined in the article “Solubility parameter values” by Eric A. Grulke in the book “Polymer Handbook”, 3rd Edition, Chapter VII, pages 519-559, by the relationship:

δ=(δd²δp²+δh²)^{1/2}

Among the hydrocarbon-based oils with a molecular weight of less than or equal to 400 g/mol and a solubility parameter δa ranging from 2 to 15 J^{0.5}/cm ^{1.2}, mention may be made of dicaprylyl ether (δa=3.45 J^{0.5}/cm^{1.5}), 2-ethylhexyl palmitate (βa=4.2 J^{0.5}/cm^{1.5}), cetyl 2-ethylhexanoate (δa=4.2 J^{0.5}/cm^{1.5}), octyldodecyl neopentanoate (δa=4.2 J^{0.5}/cm^{1.5}), isostearyl neopentanoate (δa=4.3 J^{0.5}/cm^{1.5}), isostearyl benzoate (δa=4.4 J^{0.5}/cm^{1.5}), tridecyl isononanoate (δa=4.4 J^{0.5}/cm^{1.5}), isopropyl stearate (δa=4.5 J^{0.5}/cm^{1.5}), isopropyl isostearate (δa=4.5 J^{0.5}/cm^{1.5}), isopropyl palmitate (δa=4.7 J^{0.5}cm^{1.5}), isononyl isononanoate (δa=4.87 J^{0.5}/cm^{1.5}), C₁₂₋₁₅ alkyl benzoate (δa=4.9 J^{0.5}cm^{1.5/}), isopropyl myristate (δa=5.0 J^{0.5}/cm^{1.5}), 2-ethylhexyl 2-ethylhexanoate (δa=5.2 J^{0.5}/cm^{1.5}), isodecyl neopentanoate (δa=5.3 J^{0.5}/cm ^{1.5}), 2-ethylhexyl benzoate (δa=5.9 J^{0.5}/cm^{1.5}), caprylyl carbonate (δa=6.0 J^{0.5}/cm^{1.5}) sold under the name Cetiol CC by Cognis, dioctyl (2-ethylhexyl) carbonate (δa=6.0 J^{0.5}/cm^{1.5}), diethylhexyl adipate (δa=6.2 J^{0.5}/cm^{1.5}), octadecyl 5-oxo-L-prolinate (δa=6.2 J^{0.5}/cm^{1.5}), propylene glycol dipelargonate (δa=6.4 J^{0.5}/cm^{1.5}), neopentyl glycol dicaprate (δa=6.4 J^{0.5}/cm^{1.5}), dicaprylyl maleate (δa=6.6 J^{0.5}/cm^{1.5}), propylene glycol dioctanoate (δa=6.7 J^{0.5}/cm^{1.5}), dodecyl 1-butyl-5-oxopyrrolidine-3-carboxylate (δa=7.1 J^{0.5}/cm^{1.5}), 2,4-dimethyl-1,5-pentanediol dineopentanoate (δa=7.3 J^{0.5}/cm^{1.5}), 2-octyldodecanol (δa=7.7 J^{0.5}/cm^{1.5}), isostearyl alcohol (δa=8.1 J^{0.5}/cm^{1.5}), oleyl alcohol (δa=8.2 J^{0.5}/cm^{1.5}), diisopropyl adipate (δa =8.31 J^{0.5}/cm^{1.5}), diisobutyl adipate (δa=8.31 J^{0.5}/cm^{1.5}), hexyldecanol (δa=8.6 J^{0.5}/cm^{0.5}), 2-propanyl 1-(2-ethylhexyl)-5-oxopyrrolidine-3-carboxylate (δa=8.6 J^{0.5}/cm^{1.5}), propylene glycol monoisostearate (δa=8.7 J/cm^{0.5}/cm^{1.5}), isostearyl lactate (δa=8.7 J/cm^{1.5}), butyl 1-butyl-5-oxopyrrolidine-3-carboxylate (δa=9.4 J^{0.5}/cm^{1.5}), 2-butyloctanol (δa=9.8 J^{0.5}/cm^{1.5}), C₁₂-C₁₃ alkyl lactate (δa=10.1 J^{0.5}/cm^{1.5} ), dimethyl isosorbide (δa=10.76 J^{0.5}/cm^{1.5}), tributyl citrate (δa=11.41 J^{0.5}/cm^{1.5}), triethyl citrate (δa=13.7 J^{0.5}/cm^{1.5}), phenylethyl alcohol (δa=14.0 J/cm ^{1.5}), PEG-8 (polyethylene glycol containing 8 ethylene glycol units) (δa=14.8 J^{0.5}/cm^{1.5}), and mixtures thereof.

According to a preferred embodiment, the makeup-removing composition comprises at least one hydrocarbon-based oil with a molecular weight of less than or equal to 400 g/mol and a solubility parameter βa ranging from 2 to 15 J^{0.5}/cm ^{1.5}, said oil preferably being chosen from synthetic ethers and esters, and preferably from isopropyl myristate (δa=5.0 J^{0.5}/cm^{1.5}), dicaprylyl ether (δa=3.45 J^{0.5}/cm ^{1.5}), isononyl isononanoate (δa=4.87 J^{0.5}/cm^{1.5}), isodecyl neopentanoate (δa=5.3 j^{0.5}/cm ^{1.5}), diisobutyl adipate (δa=8.31 J^{0.5}/cm^{1.5}), diisopropyl adipate (βa=8.31 J^{0.5}/cm^{1.5}), dimethyl isosorbide (δa=10.76 J^{0.5}/cm^{1.5}), tributyl citrate (δa=11.41 J^{0.5}/cm^{1.5}), and mixtures thereof.

Preferably, the makeup-removing composition comprises at least one hydrocarbon-based oil chosen from synthetic ethers containing from 10 to 40 carbon atoms, more preferentially chosen from dicaprylyl ether.

Advantageously, the total content of hydrocarbon-based oil(s) ranges from 10% to 95% by weight, preferably from 20% to 80% by weight, more preferentially from 20% to 60% by weight and better still from 25% to 40% by weight relative to the total weight of the makeup-removing composition.

The oily phase may be present in a content of between 0.1% and 80% by weight, preferably between 1% and 70% by weight, relative to the total weight of the makeup-removing composition.

Aqueous Phase

Preferably, the makeup-removing composition comprises an aqueous phase. The aqueous phase comprises water. It may also comprise at least one water-soluble organic solvent.

The term “water-soluble organic solvent” means an organic solvent that is miscible with water at 25° C.

Among the water-soluble organic solvents that may be used in the makeup-removing composition, mention may be made notably of monoalcohols containing from 1 to 5 carbon atoms such as ethanol, isopropanol and butanol, and glycols containing from 2 to 8 carbon atoms such as ethylene glycol, propylene glycol, 1,3-butylene glycol and dipropylene glycol.

Preferably, the aqueous phase (water and optionally the water-miscible solvent) is then present in the makeup-removing composition in a content ranging from 0.5% to 40% by weight, relative to the total weight of the makeup-removing composition, more preferentially ranging from 1% to 30% by weight and better still from 5% to 20% by weight relative to the total weight of the makeup-removing composition.

When the makeup-removing composition comprises water, the water content preferably ranges from 0.5% to 40% by weight, preferentially from 1% to 30% by weight and better still from 5% to 20% by weight relative to the total weight of the makeup-removing composition.

Surfactant

Preferably, the makeup-removing composition used in the context of the process according to the invention comprises c) at least one surfactant in a content of greater than or equal to 30% by weight relative to the total weight of the makeup-removing composition.

More preferentially, the surfactant(s) are nonionic.

Examples of nonionic surfactants that may be mentioned include the following nonionic surfactants:

preferably oxyalkylenated, oxyethylenated and/or oxypropylenated glycerol ethers, which may include from 10 to 150 oxyethylene and/or oxypropylene units;

oxyalkylenated alcohols, in particular oxyethylenated and/or oxypropylenated alcohols, which may include from 2 to 150 oxyethylene and/or oxypropylene units, preferably 2 to 100 oxyethylene units;

esters of a fatty acid, notably of a C₈-C₂₄ and preferably C₁₆-C₂₂ fatty acid, and of polyethylene glycol (or PEG) (which may comprise from 10 to 150 oxyethylene units), such as PEG-50 stearate and PEG-40 monostearate sold under the name Myrj 52P® by the company Uniqema;

esters of a fatty acid, notably of a C₈-C₂₄ and preferably C₁₆-C₂₂ fatty acid, and of preferably oxyalkylenated, oxyethylenated and/or oxypropylenated glycerol ethers (which may include from 10 to 150 oxyethylene and/or oxypropylene units), for instance glyceryl monostearate polyoxyethylenated with 200 oxyethylene units, sold under the name Simulsol 220 TM® by the company SEPPIC; glyceryl stearate polyoxyethylenated with 30 oxyethylene units, for instance the product Tagat S® sold by the company Goldschmidt, glyceryl oleate polyoxyethylenated with 30 oxyethylene units, for instance the product Tagat O® sold by the company Goldschmidt, glyceryl cocoate polyoxyethylenated with 30 oxyethylene units, for instance the product Varionic LI 13® sold by the company Sherex, glyceryl isostearate polyoxyethylenated with 30 oxyethylene units, for instance the product Tagat L® sold by the company Goldschmidt, and glyceryl laurate polyoxyethylenated with 30 oxyethylene units, for instance the product Tagat I® from the company Goldschmidt;

esters of a fatty acid, notably of a C₈-C₂₄ and preferably C₁₆-C₂₂ fatty acid, and of preferably oxyalkylenated, oxyethylenated and/or oxypropylenated sorbitol ethers (which may include from 2 to 150 oxyethylene and/or oxypropylene units), for instance polysorbate 60 sold under the name Tween 60® by the company Uniqema or polysorbate 21 sold under the name Tween 21-LQ® by the company Croda;

and mixtures thereof.

Preferably, the nonionic surfactant(s) c) are chosen from oxyalkylenated, in particular oxyethylenated and/or oxypropylenated, alcohols, which may include from 2 to 150 oxyethylene and/or oxypropylene units, preferably from 2 to 100 oxyethylene units, more preferentially from 2 to 50 oxyethylene units.

Preferably, the nonionic surfactant(s) c) are chosen from oxyalkylenated C₈ to C₂₄ and preferentially C₈ to C₁₈ alcohols, comprising from 2 to 100 oxyethylene units, preferably from 2 to 50 oxyethylene units, more preferentially from 2 to 40 oxyethylene units and better still from 2 to 20 oxyethylene units.

More preferentially, the nonionic surfactant c) is oxyethylenated decyl alcohol, preferably comprising 5 oxyethylene units.

Advantageously, the content of surfactant(s), in particular of nonionic surfactant(s), ranges from 30% to 80% by weight, preferably from 40% to 80% by weight, more preferentially from 50% to 70% by weight and even more preferentially from 50% to 65% by weight, relative to the total weight of the makeup-removing composition.

Additives

The makeup-removing composition may also contain any adjuvant or additive usually used.

Among the additives that may be contained in the makeup-removing composition, mention may notably be made of preserving agents, antioxidants, fragrances, mattifying fillers, cosmetic active agents, thickeners, lipophilic or hydrophilic polymers, and sequestrants.

As indicated previously, the process for treating keratin fibres according to the invention comprises the application of at least one makeup-removing composition to said keratin fibres, which have been dyed beforehand using at least one dye composition as defined above.

Preferably, the makeup-removing composition is then rinsed after an optional leave-on time, optionally followed by shampoo washing.

Preferably, the makeup-removing composition is left on for 30 seconds to 60 minutes, preferentially from 1 to 30 minutes, more preferentially from 1 to 15 minutes and better still from 2 to 10 minutes, before being rinsed out.

According to a particular embodiment, the process according to the invention also comprises a step of massaging the keratin fibres, after the application of the makeup-removing composition.

Preferably, during a massaging step, the fingers are passed along the lock ten times. The total duration of the ten passes may range from 30 seconds to 2 minutes, for example, per 1 g of lock.

The step of massaging the keratin fibres may be repeated several times, for example twice, optionally with an intermediate leave-on time.

The step of applying the makeup-removing composition may be repeated several times, optionally with intermediate rinsing. Preferably, the step of applying the makeup-removing composition is performed twice.

The time between the step of applying the keratin fibre dye composition and the step of applying the makeup-removing composition may range from a few minutes to several days, for example several tens of days. Preferably, the time between the step of applying the keratin fibre dye composition and the step of applying the makeup-removing composition ranges from 1 hour to 30 days, more preferentially from 1 day to 15 days.

Composition

The invention also relates to a makeup-removing composition, which is in the form of a microemulsion, for removing a dye composition comprising at least one colouring agent chosen from pigments, direct dyes and mixtures thereof, comprising:

a) at least one alkaline agent;
b) at least one hydrocarbon-based oil; and
c) at least one surfactant in a content of greater than or equal to 30% by weight relative to the total weight of the makeup-removing composition.

Said alkaline agent, said hydrocarbon-based oil and said surfactant are as defined above, respectively.

Preferably, said surfactant(s) are nonionic. Advantageously, said nonionic surfactant(s) are as defined above.

### Use

The invention also relates to the use of the makeup-removing composition according to the invention, or as used in the context of the process according to the invention, for removing a dye composition comprising at least one colouring agent chosen from pigments, direct dyes and mixtures thereof.

The present invention will now be described more specifically by means of examples, which do not in any way limit the scope of the invention. However, the examples make it possible to support specific features, variants and preferred embodiments of the invention.

### EXAMPLES

In the examples, the temperature is given in degrees Celsius and corresponds to room temperature (20-25° C.), unless otherwise indicated, and the pressure is atmospheric pressure, unless otherwise indicated.

The following compositions are prepared (in g/100 g, AM: Active Material):

### Dye Composition

Composition 1 below (i.e. solution of 3-aminopropyltriethoxysilane (APTES)), described in Table 1, is prepared according to the process below:

APTES (APTES Silsoft A-1100 sold by the company Momentive Performance Materials) is mixed with an aqueous solution brought to pH 1 by adding hydrochloric acid, and said mixture is placed on a VWR brand magnetic stirrer (rotation speed 500 rpm) for 24 hours at room temperature. The pH of the mixture is equal to 11.

TABLE 1

| Composition | 1 |
|---|---|
| 3-Aminopropyltriethoxysilane (APTES) | 30 |
| Aqueous hydrochloric acid solution (33.5% of | 0.02 |
| pure hydrochloric acid) | |
| Water | qs 100 |

Composition 2 below (i.e. alcoholic solution of silicone), described in Table 2, is prepared according to the process below:

the non-amino silicone compound is diluted in an ethanol solution in which the pigment (iron oxide sold by the company Sun Chemical under the name SunPuro Red Iron Oxide®) is dispersed.

TABLE 2

| Composition | 2 |
|---|---|
| Non-amino silicone (polydimethylsiloxane | 19.68 |
| (PDMS) bearing a hydroxyl terminal function | |
| (481939 sold by the company Sigma- | |
| Aldrich)) | |
| Pigment (iron oxide sold by the company Sun | 5.72 |
| Chemical under the name SunPuro Red Iron | |
| Oxide ®) | |
| Denatured ethyl alcohol | qs 100 |

Composition 1 is mixed with composition 2 in a 50/50 weight ratio. According to the above protocol, the following dye composition 3, described in Table 3, is prepared:

TABLE 3

| Composition | 3 |
|---|---|
| PDMS | 9.84 |
| Pigment | 2.86 |
| APTES | 15 |
| Aqueous hydrochloric acid solution | 0.01 |
| Water | 35 |
| Denatured ethyl alcohol | qs 100 |

### Makeup-Removing Compositions

In parallel, several makeup-removing compositions according to the invention are prepared according to the process below:

an alkaline agent is mixed with a nonionic surfactant, decyl alcohol oxyethylenated with 5 ethylene oxide units (Deceth-5), a hydrocarbon-based oil, dicaprylyl ether and water.

The microemulsion formulation is prepared by vortex stirring after all the ingredients have been mixed together.

Thus, four makeup-removing compositions according to the invention (A to D) are prepared and are described in Table 4 below:

TABLE 4

| Compositions | A | B | C | D |
|---|---|---|---|---|
| Deceth-5 | 60 | 60 | 60 | 60 |
| Dicaprylyl ether | 30 | 30 | 30 | 30 |
| Water | 9 | 9 | 9 | 9 |
| Monoethanolamine | 1 | — | — | — |
| Sodium silicate | — | 1 | — | — |
| Sodium metasilicate | — | — | 1 | — |
| Sodium hydroxide | — | — | — | 1 |

### Protocol for Dyeing Hair Locks

Composition 3 is applied to locks of dry natural hair containing 90% white hairs, at a rate of 1 g of composition per gram of lock. The locks of hair are left for 5 minutes at room temperature.

At the end of the leave-on time, the lock is dried with a hairdryer (high heat and medium power) for 3 minutes. The locks are then stored at 25° C. and 80% relative humidity for 24 hours before undergoing the makeup removal operation.

The locks of hair thus dyed then undergo a makeup removal operation with each of the compositions A to D, or a shampoo washing protocol.

### Protocol for Makeup Removal from the Locks of Hair

Each of the compositions A to D is applied to a lock of dyed hair at a rate of 3 g of composition per gram of lock. The locks of hair are massaged a first time (fingers passed along the lock ten times). Next, after a leave-on time of 5 minutes at room temperature, the locks of hair are massaged again (fingers passed along the lock ten times) and the locks of hair are then rinsed.

The locks are then washed with a standard shampoo (Gamier Ultra Doux).

The locks are then rinsed, and then blotted dry with absorbent paper.

The protocol is repeated so that two makeup removal cycles are performed.

### Shampoo Washing Protocol

The locks are washed with a standard shampoo (Gamier Ultra Doux). The locks of hair are then rinsed, combed and dried with a hairdryer.

The next shampoo wash is performed on the locks obtained after the application of the hairdryer.

The protocol is repeated so that five shampoo washes were performed on the locks of hair.

### Results

The persistence of the colour of the locks was evaluated in the CIE L*a*b* system, using a Minolta Spectrophotometer CM3600A colorimeter (illuminant D65, angle 10°, specular component included).

In this L*a*b* system, L* represents the intensity of the colour, a* indicates the green/red colour axis and b* the blue/yellow colour axis.

The persistence of the colouring is evaluated by the colour difference AE between the dyed locks before shampooing, then after having undergone five shampoo washes according to the protocol described above. The lower the AE value, the more persistent the colour with respect to shampoo washing. The results are given in Table 5 below.

The makeup-removing efficiency was also evaluated in this same CIE L*a*b* system.

The makeup-removing efficiency is evaluated by the colour difference AE between the dyed locks before shampooing, then after having undergone the makeup removal protocol described above. The higher the value of AE, the more efficient the makeup removal. The results are given in Table 6 below.

The ΔE value is calculated according to the following equation:

ΔE=\sqrt{(L*−Lₒ*)²+(a*−aₒ*)²+(b*−bₒ*)²}

In this equation, L*a*b* may represent the values measured after dyeing the hair and after performing the shampoo washes, and L0*a0*b0* may represent the values measured after dyeing the hair but before shampoo washing.

In this equation, L*a*b* may represent the values measured after dyeing the hair and after performing two makeup removal cycles, and L0*a0*b0* may represent the values measured after dyeing the hair but before the two makeup removal cycles.

TABLE 5

| | Number of | | | | |
|---|---|---|---|---|---|
| | shampoo | | | | |
| Composition | washes | L* | a* | b* | ΔE |
| 3 | 0 | 37.8 | 32.3 | 25.8 | — |
| | 5 | 39.3 | 33 | 26.1 | 1.7 |

TABLE 6

| | Makeup | | | | |
|---|---|---|---|---|---|
| Compositions | removal cycles | L* | a* | b* | ΔE |
| A | 0 | 37.8 | 32.3 | 25.8 | — |
| | 2 | 51.8 | 17.7 | 19.5 | 21.2 |
| B | 0 | 37.8 | 32.3 | 25.8 | — |
| | 2 | 45.0 | 23.1 | 20.6 | 12.8 |
| C | 0 | 37.8 | 32.3 | 25.8 | — |
| | 2 | 50.4 | 16.2 | 19.1 | 21.5 |
| D | 0 | 37.8 | 32.3 | 25.8 | — |
| | 2 | 48.1 | 21.1 | 20.8 | 16.0 |

The lock of hair dyed with dye composition 3 and washed with five successive shampoo washes has a low AE value, as indicated in Table 5.

Thus, the keratin fibres dyed using dye composition 3 show good persistence with respect to shampoo washing. Specifically, the lock of hair dyed with dye composition 3 and washed with five shampoo washes shows good persistence of the colour.

On the other hand, the locks of hair dyed with dye composition 3 and then subjected to makeup removal using compositions A to D have high AE values, in particular ΔE values that are significantly higher than the ΔE value of the lock of hair dyed with dye composition 3 and washed with five successive shampoo washes.

Thus, the value of using an alkaline agent for the removal of makeup from a lock of hair which has been dyed beforehand with dye composition 3 is observed since compositions A to D have a makeup-removing power higher than the test of the five shampoo washes.

Finally, it is observed that compositions A (monoethanolamine), C (sodium metasilicate) and D (sodium hydroxide) have a particularly improved makeup-removing power.

Thus, the process for treating keratin fibres according to the invention makes it possible to very significantly improve the removal of makeup from a lock of hair which has been dyed beforehand using a dye composition comprising at least one silicone of formula (I) and a colouring agent chosen from pigments, direct dyes and mixtures thereof.

1-17. (canceled)

18. A method for removing color from hair comprising applying to colored hair a makeup-removing composition comprising at least one alkaline agent, wherein the color removed from the hair was imparted by a dye composition comprising:
(a) at least one silicone of formula (I): 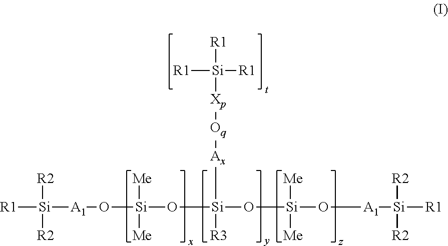
wherein:
R1 independently represents a hydroxyl group or an alkoxy group containing from 1 to 2 carbon atoms, or an alkyl group containing from 1 to 10 carbon atoms;
R2 independently represents an alkyl group containing from 1 to 10 carbon atoms, or an alkoxy group containing from 1 to 2 carbon atoms or a hydroxyl group or a monovalent radical of formula —C_{q}H_{2q}L, wherein:
q is a number ranging from 2 to 8, inclusive, and
L is an amino group which is optionally quaternized, chosen from:
—N(R″)₂, —N+(R″)₃ A⁻, —NR″-Q-N(R″)₂, or —NR″-Q-N⁺(R″)₃ A⁻,
wherein:
R″ is independent chosen from a hydrogen atom, a phenyl group, a benzyl group, or a saturated monovalent hydrocarbon-based radical;
Q denotes a linear or branched group of formula CᵣH₂ᵣ, r being an integer ranging from 2 to 6, inclusive; and
A⁻ represents a cosmetically acceptable anion;
R3 is chosen from a hydroxyl group; an alkyl group containing from 1 to 10 carbon atoms optionally substituted with at least one group chosen from a hydroxyl group (OH) or a thiol group (SH); a cycloalkyl group containing from 3 to 20 carbon atoms optionally substituted with at least one group chosen from a hydroxyl group (OH) or a thiol group (SH); an alkoxy group containing from 1 to 2 carbon atoms, optionally substituted with at least one group chosen from a hydroxyl group (OH) or a thiol group (SH); an aryl group containing from 6 to 12 carbon atoms, optionally substituted with at least one group chosen from a hydroxyl group (OH) or a thiol group (SH); a radical —(X)ₚ—Si(R₂)₃; or a monovalent radical —C_{q}H_{2q}L, as defined above;
A represents a saturated divalent hydrocarbon-based radical containing 1 carbon atom;
X represents a hydrogen atom or a saturated divalent hydrocarbon-based radical containing 1 carbon atom;
p is an integer ranging from 0 to 6, inclusive;
p′ is an integer ranging from 0 to 3, inclusive;
k is an integer ranging from 0 to 6, inclusive;
q is an integer equal to 0 or 1;
j is an integer ranging from 0 to 2, inclusive;
t is an integer equal to 0 or 1;
x denotes an integer ranging from 0 to 10, inclusive,
y denotes an integer ranging from 0 to 10, inclusive, and
z denotes an integer ranging from 0 to 500, inclusive,
with x+z ranging from 0 to 500, inclusive, and x+y+z b 4;
wherein when X represents a hydrogen atom, t=0 and p=1; and
when p=0, t=1 and q=1, and at least one of the radicals R1 or R3 denotes a hydroxyl radical or an alkoxy radical containing from 1 to 2 carbon atoms, a radical —(X)ₚ—Si(R₂)₃, or a monovalent radical of formula—C_{q}H_{2q}L, as defined above; and
(b) at least one coloring agent chosen from pigments, direct dyes, or mixtures of two or more thereof.

19. The method according to claim 18, wherein the color removed from the hair was imparted by a dye composition comprising a total amount of (a) silicones of formula (I) ranging from 0.1% to 30% by weight, relative to the weight of the dye composition.

20. The method according to claim 18, wherein the color removed from the hair was imparted by a dye composition further comprising at least one alkoxysilane chosen from the compounds of formula (II), oligomers thereof, or mixtures thereof:
(R¹ₓ)—SH—OR²)₍₄₋ₓ₎ (II)
wherein:
R¹ represents an alkoxy group containing from 1 to 10 carbon atoms, a linear or branched, saturated or unsaturated, cyclic or acyclic C₁ to C₂₂ hydrocarbon-based radical, optionally substituted with at least one group chosen from a hydroxyl group (OH); a thiol group; an amino group NH₂; an alkylamino group NH-R wherein R denotes a linear or branched alkyl radical containing from 1 to 20 carbon atoms; an alkoxy group containing from 1 to 10 carbon atoms; a cycloalkyl containing from 3 to 40 carbon atoms; or an aryl containing from 6 to 30 carbon atoms;
R² represents a hydrogen atom or an alkyl group containing from 1 to 20 carbon atoms; and
x denotes an integer ranging from 1 to 3.

21. The method according to claim 18, wherein the color removed from the hair was imparted by a dye composition comprising a total amount of (b) coloring agents ranging from 0.001% to 20% by weight, relative to the weight of the dye composition.

22. The method according to claim 21, wherein the color removed from the hair was imparted by a dye composition comprising at least one pigment.

23. The method according to claim 22, wherein the total amount of pigments in the dye composition ranges from 0.05% to 20% by weight, relative to the weight of the dye composition.

24. The method according to claim 22, wherein the total amount of pigments in the dye composition ranges from 0.1% to 15% by weight, relative to the weight of the dye composition.

25. The method according to claim 18, wherein the color removed from the hair was imparted by a dye composition comprising at least one direct dye.

26. The method according to claim 25, wherein the total amount of direct dyes in the dye composition ranges from 0.001% to 10% by weight, relative to the weight of the dye composition.

27. The method according to claim 25, wherein the total amount of direct dyes in the dye composition ranges from 0.005% to 5% by weight, relative to the weight of the dye composition.

28. The method according to claim 18, wherein the make-up removing composition comprises at least one alkaline agent chosen from alkanolamines, alkali metal silicates, metasilicates, mineral hydroxides, or mixtures of two or more thereof.

29. The method according to claim 18, wherein the total amount of alkaline agents in the make-up removing composition ranges from 0.01% to 15% by weight, relative to the weight of the makeup-removing composition.

30. The method according to claim 18, wherein the makeup-removing composition is in the form of a microemulsion.

31. The method according to claim 18, wherein the makeup-removing composition further comprises at least one hydrocarbon-based oil.

32. The method according to claim 31, wherein the total amount of hydrocarbon-based oils in the make-up removing composition ranges from 10% to 95% by weight, relative to the weight of the makeup-removing composition.

33. The method according to claim 18, wherein the makeup-removing composition further comprises at least one surfactant in an amount greater than or equal to 30% by weight, relative to the weight of the makeup-removing composition.

34. The method according to claim 33, wherein the at least one surfactant is nonionic.

35. The method according to claim 33, wherein the at least one surfactant is chosen from oxyalkylenated alcohols having from 2 to 150 oxyethylene and/or oxypropylene units.

36. The method according to claim 33, wherein the total amount of surfactants in the make-up removing composition ranges from 30% to 80% by weight, relative to the weight of the makeup-removing composition.

37. A makeup-removing composition for removing a dye chosen from pigments and/or direct dyes, the make-up removing composition comprising:
a) at least one alkaline agent;
b) at least one hydrocarbon-based oil; and
c) at least one surfactant in a content of greater than or equal to 30% by weight relative to the weight of the makeup-removing composition,
wherein the make-up removing composition is in the form of a microemulsion.
